(19) United States
(12) Patent Application Publication
(10) Pub. No.: US 2006/0009509 A1
(43) Pub. Date: Jan. 12, 2006

(54) Progesterone receptor antagonists, contraceptive regimens, and kits

(75) Inventors: Gary Sondermann Grubb, Newtown Square, PA (US); Ginger Dale Constantine, Malvern, PA (US); Andrew Fensome, Wayne, PA (US); Casey Cameron McComas, Phoenixville, PA (US); Edward George Melenski, Collegeville, PA (US); Michael Anthony Marella, Limerick, PA (US); Jay Edward Wrobel, Lawrenceville, NJ (US)

Correspondence Address: HOWSON AND HOWSON;CATHY A. KODROFF, ONE SPRING HOUSE CORPORATE CENTER, BOX 457, SPRING HOUSE, PA, 19477, US

(73) Assignee: Wyeth, Madison, NJ (US)

(21) Appl. No.: 11/175,549

(22) Filed: Jul. 6, 2005

Related U.S. Application Data
(60) Provisional application No. 60/676,135, filed on Apr. 29, 2005.
(60) Provisional application No. 60/585,883, filed on Jul. 7, 2004.

(51) Int. Cl. A61K 31/404

(52) U.S. Cl. 514/414

(57) ABSTRACT

A method of contraception is provided which involves delivery of 21 to 27 consecutive days of one or more PR antagonists in the absence of a progestin, estrogen, or other steroidal compound, followed by 1 to 7 days without any active agent. Also described is a pharmaceutically useful kit to facilitate delivery of this regimen.

### CROSS-REFERENCE TO RELATED APPLICATIONS

This application claims the benefit of the priorities of U.S. Provisional Patent Application No. 60/676,135, filed Apr. 29, 2005 and U.S. Provisional Patent Application No. 60/585,883, filed Jul. 7, 2004.

### BACKGROUND OF THE INVENTION

The use of a progesterone receptor (PR) antagonist (RU-486 or mifepristone) as a contraceptive was first reported in 1985 with very little research reported on other PR antagonists as a contraceptive. Over the past 19 years, the research has been carried on by academic groups and not-for-profit agencies (e.g., World Health Organization (WHO) and National Institutes of Health (NIH)). There has been a trend toward the use of lower doses of mifepristone. While many different regimens have been tested, the continuous administration of mifepristone has been the most reliable for inhibiting ovulation.

The efficacy of continuous dosing of mifepristone has been evaluated by assessing ovulation rates. Continuous administration of low doses of mifepristone (2-10 mg) has been shown to prevent ovulation in two studies: Spitz I M, et al, Fert & Steril. 59 (5):971-975, (May 1993) and Ledger et al, Hu Reprod, 7(7):945-950 (August 1992) over one treatment cycle with a small number of subjects of about 5 per dose group. A continuous dose of 1 mg appeared to prevent ovulation in one study of 11 subjects (Batista et al, Am J Obstet. Gynecol. 167(10): 60-65 (July 1992)) but not in another study with 5 subjects (Croxatto et al, Hum Reprod., 8(1):201-207 (February 1993)). In the first study (Croxatto et al, Hum Reprod, 13(4):793-798 (April 1998)) to administer mifepristone continuously for longer than one cycle, 14 of 21 women ovulated at least once during the 3-month treatment with 1 mg mifepristone daily.

The second study of mifepristone given continuously to inhibit ovulation was done with 2 and 5 mg of mifepristone (Brown et al, J. Clin. Endocrinol. Metab. 87(1): 63-70 (January 2002)) at two clinical sites. In the Edinburgh site, ovulations occurred in 9.6% and 5.2% of cycles with 2 and 5 mg, respectively, over 4 months of treatment. In the Shanghai clinical site, ovulations occurred in 2.5% and 1.2% of cycles with 2 and 5 mg, respectively. These low ovulation rates are comparable to those seen for some low-dose standard oral contraceptives (OCs) containing a progestin and estrogen. Since these standard OCs are given in a 21-day cycle followed by 7 days of placebo, it is expected that a PR antagonist with a low ovulation rate could also be administered in a similar regimen and provide good contraceptive effectiveness.

When given for more than one cycle, a continuous regimen of mifepristone increases the occurrence of amenorrhea. Brown et al, cited above (2002) found that in subjects treated for 4 months with 2 and 5 mg of mifepristone, the occurrence of amenorrhea was 65% and 88%, respectively, at the Edinburgh site and 90% for both doses, at the Shanghai site. All subjects reported a menstrual bleed within 3 weeks after stopping the 4 months of treatment. This high rate of amenorrhea is unacceptable to many women who want to have a monthly menstrual bleed.

What is needed are methods of contraception that avoid amenorrhea.

### SUMMARY OF THE INVENTION

In one aspect, the invention provides a contraceptive regimen that involves delivery of a PR antagonist as the sole active agent for 21 to 27 days consecutively followed by 1 to 7 days in which no effective amount of an active agent is delivered. During these 1 to 7 days, a placebo may be administered. Generally, within 2 to 3 days following the completion of the first phase (within the period in which no PR antagonist is delivered), menstruation occurs.

In a further aspect, the invention provides pharmaceutically useful kits for administering the regimen and compounds of the invention.

Other aspects and advantages of the invention will be readily apparent from the following detailed description of the invention.

### DETAILED DESCRIPTION OF THE INVENTION

In one aspect, the present invention provides a method of contraception in a female of child-bearing age. This method is particularly useful for females seeking to avoid amenorrhea. In this method, an PR antagonist, or combination of PR antagonists, is delivered for a period of consecutive days as the sole active (i.e., anti-contraceptive) agent in order to prevent ovulation.

A PR antagonist can be any compound that binds to the PR receptor and inhibits the activity of progestational agents. In this disclosure, the terms anti-progestational agents, and progesterone receptor antagonists are understood to be synonymous.

Examples of PR antagonists that are useful in contraception and in the contraceptive regimens of the invention include compounds of formula I: 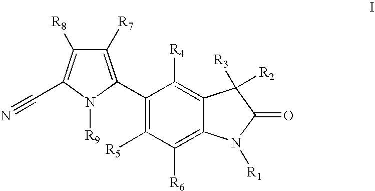
wherein, R₁ is hydrogen, alkyl, substituted alkyl, cycloalkyl, C₃-C₆ alkenyl, or C₃-C₆ alkynyl; R₂ and R₃ are independently selected from among hydrogen, alkyl or substituted alkyl; or R₂ and R₃ are taken together to form a ring and together contain —CH₂—(CH₂)ₙ—CH₂— where n is 0 (i.e., a chemical bond), 1, 2, or 3; R₄ is hydrogen or halogen; R₅ is hydrogen; R₆ is hydrogen or halogen; R₇ is hydrogen, alkyl, or halogen; R₈ is hydrogen; R₉ is hydrogen, alkyl, substituted alkyl, or COOR^{A}; and R^{A} is alkyl, or substituted alkyl; or a pharmaceutically acceptable salt, a prodrug, or a tautomer thereof.

In one embodiment, R₁ is hydrogen or alkyl and R₂ and R₃ are taken together to form a ring and together contain —CH₂—(CH₂)ₙ—CH₂— where n is 1 or 2. In another embodiment, R₂ or R₃, or both, are a C₁ to C₆ alkyl. For example, either R₂ or R₃, or both, can be ethyl. In another example, R₂ or R₃, or both, are methyl. In another embodiment, R₉ is a substituted or unsubstituted C₁ to C₆ alkyl. For example, R₉ can be methyl or ethyl. In another example, R₉ is C₁ to C₂ substituted with a phenyl. In still another embodiment, R₉ is COOR^{A}. In one example, R^{A} is tert-butyl.

In another embodiment, where the structure contains a halogen, the halogen is a F. However, other halogens, e.g., Cl, I or Br, may be selected. In one embodiment, R₆ is F. In another embodiment, R₄ is F.

In a further embodiment, where R₁ and/or R₉ are substituted alkyl, the alkyl is substituted with a halogen, nitrile or benzene ring. In one embodiment, where R₁ is a cycloalkyl, it is C₃ to C₆ alkyl.

In still another embodiment, the PR antagonist is 5-(7-fluoro-3,3-dimethyl-2-oxo-2,3-dihydro-1H-indol-5-yl)-1-methyl-1H-pyrrole-2-carbonitrile, 5-(4-fluoro-3,3-dimethyl-2-oxo-2,3-dihydro-1H-indol-5-yl)-1-methyl-1H-pyrrole-2-carbonitrile, 5-(7′-fluoro-2′-oxo-1′,2′-dihydrospiro[cyclopropane-1,3′-indol]-5′-yl)-1-methyl-1H-pyrrole-2-carbonitrile, 5-(7-fluoro-2-oxo-1,3,3-trimethyl-2-oxo-2,3-dihydro-1H-indol-5-yl)-1-methyl-1H-pyrrole-2-carbonitrile, 5-(7-fluoro-3,3-dimethyl-2-oxo-2,3-dihydro-1H-indol-5-yl)-1H-pyrrole-2-carbonitrile, tert-butyl-2-cyano-5-(7-fluoro-3,3-dimethyl-2-oxo-2,3-dihydro-1H-indol-5-yl)-1H-pyrrole-1-carboxylate, Methyl-[5-(5-cyano-1-methyl-1H-pyrrol-2-yl)-7-fluoro-3,3-dimethyl-2-oxo-2,3-dihydro-1H-indol-1-yl]acetate, 5-(1-ethyl-7-fluoro-3,3-dimethyl-2-oxo-2,3-dihydro-1H-indol-5-yl)-1-methyl-1H-pyrrole-2-carbonitrile, 5-(7-fluoro-3,3-dimethyl-2-oxo-1-prop-2-yn-1-yl-2,3-dihydro-1H-indol-5-yl)-1-methyl-1H-pyrrole-2-carbonitrile, 5-[7-fluoro-3,3-dimethyl-2-oxo-1(2-phenylethyl)-2,3-dihydro-1H-indol-5-yl]-1-methyl-1H-pyrrole-2-carbonitrile, 5-(1-benzyl-7-fluoro-3,3-dimethyl-2-oxo-2,3-dihydro-1H-indol-5-yl)-1-methyl-1H-pyrrole-2-carbonitrile, 5-(7-fluoro-3,3-dimethyl-2-oxo-1-propyl-2,3-dihydro-1H-indol-5-yl)-1-methyl-1H-pyrrole-2-carbonitrile, 5-(7-fluoro-1-isobutyl-3,3-dimethyl-2-oxo-2,3-dihydro-1H-indol-5-yl)-1-methyl-1H-pyrrole-2-carbonitrile, 5-(7-fluoro-1-isopropyl-3,3-dimethyl-2-oxo-2,3-dihydro-1H-indol-5-yl)-1-methyl-1H-pyrrole-2-carbonitrile, 5-(1-allyl-7-fluoro-3,3-dimethyl-2-oxo-2,3-dihydro-1H-indol-5-yl)-1-methyl-1H-pyrrole-2-carbonitrile, 5-(1-cyclohexyl-7-fluoro-3,3-dimethyl-2-oxo-2,3-dihydro-1H-indol-5-yl)-1-methyl-1H-pyrrole-2-carbonitrile, 5-(1-cyclopentyl-7-fluoro-3,3-dimethyl-2-oxo-2,3-dihydro-1H-indol-5-yl)-1-methyl-1H-pyrrole-2-carbonitrile, or a pharmaceutically acceptable salt, tautomer, or prodrug thereof.

The compounds of formula I are prepared by coupling an oxindole with a substituted pyrrole. Specifically, these compounds can be prepared by (a) alkylating a substituted oxindole; (b) brominating the product of (a); and coupling the product of (b) with a substituted pyrrole.

Desirably, the compounds of formula I are readily prepared by one of skill in the art according to the following schemes from commercially available starting materials or starting materials which can be prepared using literature procedures. These schemes show the preparation of representative compounds of this invention. Variations on these methods, or other methods known in the art, can be readily utilized by one of skill in the art given the information provided herein.

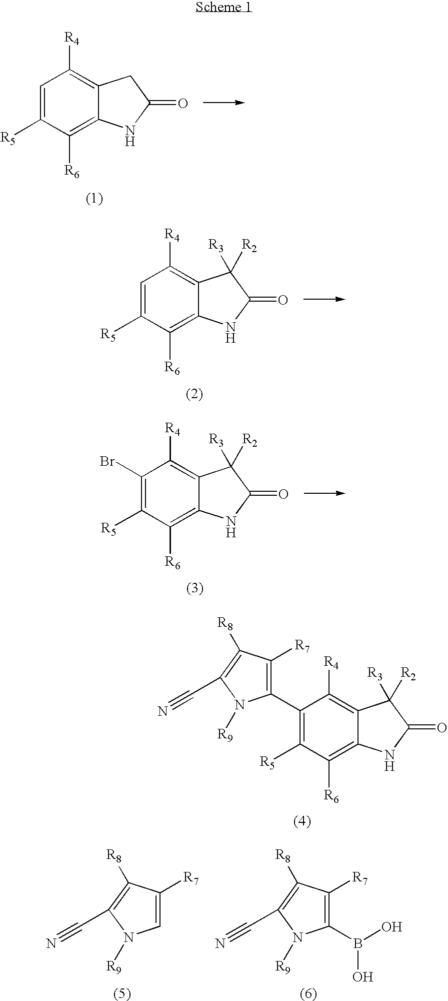

According to scheme 1, an appropriately substituted oxindole (1) is treated with a suitable base (normally 2 or more molar equivalents) and an alkylating agent to afford substituted oxindoles (2). The range of suitable bases includes alkyl lithium bases, potassium tertiary butoxide, sodium hexamethyldisilazide and similar bases. The base may also be used in conjunction with an additive. Generally the compounds of the invention were prepared using n-butyl lithium as the base in anhydrous tetrahydrofuran (THE) in the presence of lithium chloride. The alkylating agent is normally an alkyl halide (e.g., bromide or iodide) but could also be a triflate, tosylate or mesylate. If one equivalent of alkylating agent is used then the resultant oxindole will be mono-substituted. With two equivalents, then the oxindole will be di-substituted. If the alkylating agent is bifunctional (e.g., a halide or other leaving group at both ends of an alkyl chain) then a spirocyclic ring is produced.

Oxindoles (2) are then brominated to give compound (3). The bromination is conveniently carried out with bromine in a solvent such as methylene chloride or acetic acid, which may be buffered with an additive such as sodium acetate. The bromination may also be accomplished with N-bromosuccinimide or pyridinium bromide per bromide. Compound (3) is then converted into compound (4) under the action of a palladium catalyst and a suitable coupling partner. The coupling partner may be formed in situ from the pyrrole (5) and lithium di-isopropylamide and a trialkyl borate or may be the pre-formed boronic acid (6). The source of palladium is normally tetrakis(triphenylphosphine)palladium (0) or another suitable source such as palladium dibenzylidene acetone in the presence of tributylphosphine (Fu, G. C. et al. Journal of the American Chemical Society, 2000, 122, 4020, for alternate catalyst systems see also Hartwig, J. F. et al. Journal of Organic Chemistry, 2002, 67, 5553). A base is also required in the reaction; the normal choices are sodium or potassium carbonate, cesium fluoride, potassium fluoride, potassium phosphate or a tertiary amine base such as triethylamine. The choice of solvents includes THF, dimethoxy ethane (DME), dioxane, ethanol, water, and toluene amongst others. Depending on the reactivity of the coupling partners and reagents, the reaction may be conducted up to the boiling point of the solvents, or may indeed be accelerated under microwave irradiation, if necessary.

Alternatively, compounds (1) to (3) can be prepared according to the routes described in U.S. Provisional Patent Application Nos. 60/676,149 and 60/676,381, which are hereby incorporated by reference in their entirety.

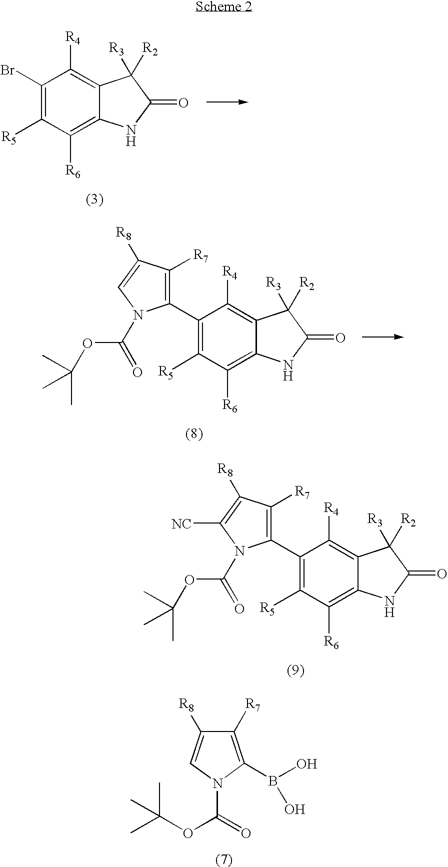

An alternative strategy may be used when R₉=hydrogen, scheme 2. Thus the bromide (3) is coupled with a pyrrole boronic acid of formula (7) under conditions as described above. Compound (8) may then be converted into the nitrile (9). This is most conveniently accomplished by the action of chlorosulfonylisocyanate followed by treatment with DMF, although other methods are also available. The t-butylcarbonate protecting group is then removed to afford the product (4), R₉=H.

When R₁ is to be a substituted alkyl group, then compound (4) is treated with a suitable base (for example sodium hydride, potassium tert-butoxide or cesium carbonate) in a solvent such as THF or DMF, followed by treatment with the appropriate alkylating agent. The alkylating agent would normally be an alkyl halide, or an alkyl sulfonate (tosylate, mesylate or triflate for example).

Other examples of PR antagonists useful in the invention include mifepristone, onapristone, lilopristone (M. Bygdeman et al, Acta Obstet. Gynecol. Scand., Suppl. 1997, 164:75-7), asoprisinil (D. Demanno et al, Steroids, 68(10-13):1019-1032 (November 2003); K. Schwalisz et al, Semin Reprod Med (May 2004); 22(2):113-9), and CDB-2914 (P. Stratton et al. Hu Reproduction, 15(5):1092-1099 (May 2000)).

In one desirable embodiment, the PR antagonist used in the regimens and kits of the invention is 3-Chloro-5-(4,4-dimethyl-2-oxo-1,4-dihydro-2H-benzo[d][1,3]oxazin-6-yl)-benzonitrile which has the formula: 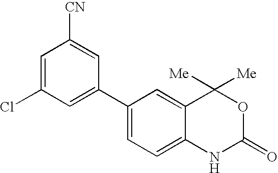
or pharmaceutically acceptable salts, esters, or other prodrug forms thereof. This compound and methods of producing same have been described in U.S. Pat. Nos. 6,566,358; 6,509,334; and 6,713,478, which are incorporated by reference herein.

In another desirable embodiment, the PR antagonist is 5-(7-fluoro-3,3-dimethyl-2-oxo-2,3-dihydro-1H-indol-5-yl)-1-methyl-1H-pyrrole-2-carbonitrile, which has the formula: 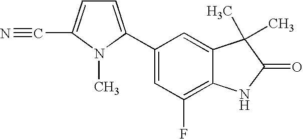
or pharmaceutically acceptable salts, esters, or other prodrug forms thereof.

In still a further embodiment, one of skill in the art can utilize a PR antagonist. of Formula II: 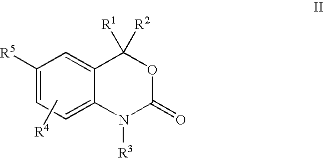
wherein:

R¹ and R² are independent substituents selected from among H, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heterocyclic, substituted heterocyclic, COR^{A}, and NR^{B}COR^{A};

or R¹ and R² are fused to form:

a) an optionally substituted 3 to 8 membered saturated spirocyclic ring;

b) an optionally substituted 3 to 8 membered spirocyclic ring having one or more carbon-carbon double bonds; or

c) an optionally substituted 3 to 8 membered heterocyclic ring containing one to three heteroatoms selected from among O, S and N; the spirocyclic rings of a), b) and c) being optionally substituted by from 1 to 4 groups selected from among fluorine, C₁ to C₆ alkyl, C₁ to C₆ alkoxy, C₁ to C₆ thioalkyl, —CF₃, —OH, —CN, NH₂, —NH(C₁ to C₆ alkyl), and —N(C₁ to C₆ alkyl)₂;

R^{A} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;

R^{B} is H, C₁ to C₃ alkyl, or substituted C₁ to C₃ alkyl;

R³ is H, OH, NH₂, C₁ to C₆ alkyl, substituted C₁ to C₆ alkyl, C₃ to C₆ alkenyl, substituted C₃ to C₆ alkenyl, alkynyl, substituted alkynyl, or COR^{C};

R^{C} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;

R⁴ is H, halogen, CN, NO₂, C₁ to C₆ alkyl, substituted C₁ to C₆ alkyl, alkynyl, or substituted alkynyl, C₁ to C₆ alkoxy, substituted C₁ to C₆ alkoxy, amino, C₁ to C₆ aminoalkyl, or substituted C₁ to C₆ aminoalkyl;

R⁵ is selected from among a) and b):

a) a substituted benzene ring containing the substituents X, Y and Z as shown below: 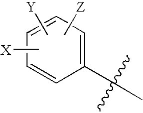
wherein:

X is selected from among halogen, CN, C₁ to C₃ alkyl, substituted C₁ to C₃ alkyl, alkenyl, substituted alkenyl, alkynyl, substituted alkynyl, C₁ to C₃ alkoxy, substituted C₁ to C₃ alkoxy, C₁ to C₃ thioalkoxy, substituted C₁ to C₃ thioalkoxy, amino, C₁ to C₃ aminoalkyl, substituted C₁ to C₃ aminoalkyl, NO₂, C₁ to C₃ perfluoroalkyl, 5 or 6 membered heterocyclic ring containing in its backbone 1 to 3 heteroatoms, COR^{D}, OCOR^{D}, and NR^{E}COR^{D};

R^{D} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;

R^{E} is H, C₁ to C₃ alkyl, or substituted C₁ to C₃ alkyl;

Y and Z are independent substituents selected from among H, halogen, CN, NO₂, amino, aminoalkyl, C₁ to C₃ alkoxy, C₁ to C₃ alkyl, and C₁ to C₃ thioalkoxy; or

b) a five or six membered ring having in its backbone 1, 2, or 3 heteroatoms selected from among O, S, SO, SO₂ or NR⁶ and containing one or two independent substituents selected from H, halogen, CN, NO₂, amino, C₁ to C₃ alkyl, C₁ to C₃ alkoxy, C₁ to C₃ aminoalkyl, COR^{F}, and NR^{G}COR^{F};

R^{F} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;

R^{G} is H, C₁ to C₃ alkyl, or substituted C₁ to C₃ alkyl;

R⁶ is H or C₁ to C₃ alkyl;

or pharmaceutically acceptable salt thereof.

In another embodiment, the compounds used in this invention are characterized by formula I, wherein:

R¹=R² and are CH₃; or

R¹ and R² are a saturated spirocyclic ring constructed by fusing R¹ and R² to form a 6 membered spirocyclic ring;

R³ is H, OH, NH₂, CH₃, substituted CH₃, or COR^{C};

R^{C} is H, C₁ to C₃ alkyl, or C₁ to C₄ alkoxy;

R⁴ is H, halogen, NO₂, CN, or C₁ to C₃ alkyl;

R⁵ is a disubstituted benzene ring containing the substituents X and Y as shown below:

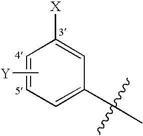

X is selected from among halogen, CN, methoxy, NO₂, and 2-thiazole;

Y is H or F; or

R⁵ is a five membered ring with the structure:

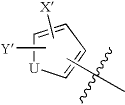

U is O, S, or NH;

X′ is halogen, CN, or NO₂, provided that when U is NR⁶, X′ is not CN;

Y′ is H or C₁ to C₄ alkyl

and pharmaceutically acceptable salts.

In a further embodiment, the 1,4-dihydro-benzo[d][1,3]oxazin-2-one compounds of U.S. Pat. Nos. 6,509,334; 6,566,358; and 6,713,478 are useful in the invention.

Other suitable compounds for use in the present invention include, e.g., the 1,3-dihydro-indol-2-one compounds of U.S. Pat. No. 6,391,907, the 2,3-dihydro-1H-indole compounds of U.S. Pat. No. 6,417,214, the benzimidazolones and analogues thereof described in U.S. Pat. No. 6,380,235, the 2,1-benzisothiazoline 2,2-dioxides of U.S. Pat. No. 6,339,098, the cyclocarbamates and cyclo amides described in U.S. Pat. Nos. 6,306,851 and 6,441,019, the cyclic urea and cyclic amide derivatives described in U.S. Pat. No. 6,369,056, and the quinazolinone and benzoxazine derivatives described in U.S. Pat. No. 6,358,948. Still other suitable compounds for use in the present invention include, e.g., ORG-31710, ORG-31376, ORG-33832, ORG-33245, ORG-33628, ORG-31806, RU-2992, RU-1479, RU-25056, RU-49295; Mifepristone/RU-486; RU-46556; CDB-4124; J-956; Asoprisnil/J-867; J-900; RWJ-26819; LG1127; LG120753; LG120830; LG1447; LG121046; CDB-2914; CGP-19984A; RTI-3021-012; RWJ-25333; ZK-112993; ZK-136796; ZK-114043; Onapristone/ZK-28299; Lilopristone/ZK-98734; ZK-230211; ZK-136798; and ZK-137316.

Examples of other suitable PR antagonists may be found in U.S. Pat. Nos. 6,391,907; 6,608,086; 6,417,214; 6,380,235; 6,339,098; 6,306,851; 6,369,056; and 6,358,948.

The term “alkyl” is used herein to refer to both straight- and branched-chain saturated aliphatic hydrocarbon groups having one to eight carbon atoms, desirably one to six carbon atoms (i.e., C₁, C₂, C₃, C₄, C₅ or C₆); “alkenyl” is intended to include both straight- and branched-chain alkyl groups with at least one carbon-carbon double bond and two to eight carbon atoms, desirably two to six carbon atoms; “alkynyl” group is intended to cover both straight- and branched-chain alkyl groups with at least one carbon-carbon triple bond and two to eight carbon atoms, desirably two to six carbon atoms.

The terms “substituted alkyl”, “substituted alkenyl”, and “substituted alkynyl” refer to alkyl, alkenyl, and alkynyl as just described having from one to three substituents selected from the group including halogen, CN, OH, NO₂, amino, aryl, heterocyclic, substituted aryl, substituted heterocyclic, alkoxy, aryloxy, substituted alkyloxy, alkylcarbonyl, alkylcarboxy, alkylamino, or arylthio. These substituents may be attached to any carbon of an alkyl, alkenyl, or alkynyl group provided that the attachment constitutes a stable chemical moiety.

The term “acyl” as used herein refers to a carbonyl substituent, i.e., a C(O)(R) group where R is a straight- or branched-chain saturated aliphatic hydrocarbon group including, without limitation, alkyl, alkenyl, and alkynyl groups. Desirably, the R groups have 1 to about 8 carbon atoms, and more desirably 1 to about 6 carbon atoms. The term “substituted acyl” refers to an acyl group which is substituted with 1 or more groups including halogen, CN, OH, and NO₂.

The term “aryl” is used herein to refer to an aromatic system which may be a single ring or multiple aromatic rings fused or linked together as such that at least one part of the fused or linked rings forms the conjugated aromatic system. The aryl groups include, but are not limited to, phenyl, naphthyl, biphenyl, anthryl, tetrahydronaphthyl, and phenanthryl.

The term “substituted aryl” refers to aryl as just defined having one to four substituents selected from among halogen, CN, OH, NO₂, amino, alkyl, cycloalkyl, alkenyl, alkynyl, alkoxy, aryloxy, substituted alkyloxy, alkylcarbonyl, alkylcarboxy, alkylamino, or arylthio.

The term “heterocyclic” is used herein to describe a stable 4- to 7-membered monocyclic or a stable multicyclic heterocyclic ring which is saturated, partially unsaturated, or unsaturated, and which consists of carbon atoms and from one to four heteroatoms selected from among N, O, and S atoms. The N and S atoms may be oxidized. The heterocyclic ring also includes any multicyclic ring in which any of above defined heterocyclic rings is fused to an aryl ring. The heterocyclic ring may be attached at any heteroatom or carbon atom provided the resultant structure is chemically stable. Such heterocyclic groups include, without limitation, tetrahydrofuran, piperidinyl, piperazinyl, 2-oxopiperidinyl, azepinyl, pyrrolidinyl, imidazolyl, pyridyl, pyrazinyl, pyrimidinyl, pyridazinyl, oxazolyl, isoxazolyl, morpholinyl, indolyl, quinolinyl, thienyl, furyl, benzofuranyl, benzothienyl, thiamorpholinyl, thiamorpholinyl sulfoxide, and isoquinolinyl.

The term “substituted heterocyclic” is used herein to describe the heterocyclic just defined having one to four substituents selected from among, without limitation, halogen, CN, OH, NO₂, amino, alkyl, substituted alkyl, cycloalkyl, alkenyl, substituted alkenyl, alkynyl, alkoxy, aryloxy, substituted alkyloxy, alkylcarbonyl, alkylcarboxy, alkylamino, or arylthio.

The term “arylthio” as used herein refers to the S(aryl) group, where the point of attachment is through the sulfur-atom and the aryl group can be optionally substituted. The term “alkoxy” is used herein to refer to the OR group, where R is alkyl or substituted alkyl. The term “aryloxy” is used herein to refer to the OR group, where R is aryl or substituted aryl. The term “alkylcarbonyl” is used herein to refer to the RCO group, where R is alkyl or substituted alkyl. The term “alkylcarboxy” is used herein to refer to the COOR group, where R is alkyl or substituted alkyl. The term “aminoalkyl” refers to both secondary and tertiary amines wherein the alkyl or substituted alkyl groups, containing one to eight carbon atoms, which may be either same or different and the point of attachment is on the nitrogen atom. The term “halogen” refers to Cl, Br, F, or I.

The compounds of the present invention can contain one or more asymmetric centers and can thus give rise to optical isomers and diastereomers. While shown without respect to stereochemistry, the compounds can include optical isomers and diastereomers; racemic and resolved enantiomerically pure R and S stereoisomers; other mixtures of the R and S stereoisomers; and pharmaceutically acceptable salts thereof.

The compounds of the present invention can also encompass tautomeric forms of the structures provided herein characterized by the bioactivity of the drawn structures. Further, the compounds of the present invention can be used in the form of salts derived from pharmaceutically or physiologically acceptable acids or bases.

Pharmaceutically acceptable salts can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids. Salts may also be formed from inorganic bases, desirably alkali metal salts, for example, sodium, lithium, or potassium, and organic bases, such as ammonium, mono-, di-, and trimethylammonium, mono-, di- and triethylammonium, mono-, di- and tripropyl-ammonium (iso and normal), ethyldimethylammonium, benzyldimethylammonium, cyclohexylammonium, benzylammonium, dibenzylammonium, piperidinium, morpholinium, pyrrolidinium, piperazinium, 1-methylpiperidinium, 4-ethylmorpholinium, 1-isopropylpyrrolidinium, 1,4-dimethylpiperazinium, 1-n-butyl piperidinium, 2-methylpiperidinium, 1-ethyl-2-methylpiperidinium, mono-, di- and triethanolammonium, ethyl diethanolammonium, n-butylmonoethanolammonium, tris(hydroxymethyl)methylammonium, phenylmonoethanolammonium, and the like.

Physiologically acceptable alkali salts and alkaline earth metal salts can include, without limitation, sodium, potassium, calcium and magnesium salts in the form of esters, and carbamates. Other conventional “pro-drug” forms can also be utilized which, when delivered in such form, convert to the active moiety in vivo.

These salts, as well as other compounds of the invention, can be in the form of esters, carbamates and other conventional “pro-drug” forms, which, when administered in such form, convert to the active moiety in vivo. In a currently preferred embodiment, the prodrugs are esters. See, e.g., B. Testa and J. Caldwell, “Prodrugs Revisited: The “Ad Hoc” Approach as a Complement to Ligand Design”, Medicinal Research Reviews, 16(3):233-241, ed., John Wiley & Sons (1996).

The compounds discussed herein also encompass “metabolites” which are unique products formed by processing the compounds of the invention by the cell or patient. Desirably, metabolites are formed in vivo.

The method of the invention is performed for a period of time corresponding to the length of a menstrual cycle, i.e., in the range of 23 to 35 days, with 28 days being the average. Thus, the method of the invention involves delivering a daily dosage unit containing an effective amount of an active agent consisting of an PR antagonist to a female of child bearing age over a period of 18 to 28 consecutive days followed by 1 to 7 consecutive days in which no effective amount of an active agent is delivered to the subject.

The term “effective amount” of a PR antagonist(s) is a dosage that prevents contraception. Without being bound by theory, this is achieved primarily by preventing ovulation. The term “no effective amount” of a PR antagonist(s) is used to refer to the 1 to 7 days following delivery of an effective amount of the PR antagonist(s). During this period, desirably, no amount of a PR antagonist(s) is delivered to the animal. However, it is possible, depending upon the delivery route, that a sustained release formulation may be “leaky” and continue to deliver low amounts of a PR antagonist which are not effective at contraception during this period. The phrase “no effective amount” encompasses delivery of no amount of PR antagonist(s).

According to the present invention, a female is a desirably a human. However, as used herein, a female can include non-human mammals, e.g., cattle or livestock, horses, pigs, domestic animals, etc.

In one aspect, the method of invention involves delivering a daily dosage unit containing an active agent for 28 consecutive days. In the embodiment, the regimen consists of delivering a PR antagonist to a female of child-bearing age over a period of 21 to 27 consecutive days followed by 1 to 7 consecutive days in which no effective amount or no amount of active agent is delivered to the subject. Optionally, the period of 1 to 7 days in which no effective amount of an active agent is delivered to the subject can involve delivery of a second phase of daily dosage units of 1 to 7 days of a pharmaceutically acceptable placebo. Alternatively, during this “placebo period”, no placebo is administered.

In one embodiment, the method of the invention involves delivering a PR antagonist as the sole active agent for 21 consecutive days followed by 7 days in which no effective amount of an active agent is delivered. Optionally, during these 7 days, a second phase of 7 daily dosage units of an orally and pharmaceutically acceptable placebo can be delivered.

In another embodiment, the method of the invention involves delivering a PR antagonist as the sole active agent for 23 consecutive days followed by 5 days in which no effective amount of an active agent is delivered. Optionally, during these 5 days, a second phase of 5 daily dosage units of an orally and pharmaceutically acceptable placebo can be delivered.

In a further embodiment, the method of the invention involves delivering a PR antagonist as the sole active agent for 25 consecutive days followed by 3 days in which no effective amount of an active agent is delivered. Optionally, during these 3 days, a second phase of 3 daily dosage units of an orally and pharmaceutically acceptable placebo can be delivered.

In still another embodiment, the method of the invention involves delivering a PR antagonist as the sole active agent for 27 consecutive days followed by 1 day in which no effective amount of an active agent is delivered. Optionally, a second phase of 1 daily dosage unit of an orally and pharmaceutically acceptable placebo can be delivered.

This invention also includes the use of pharmaceutical compositions containing one or more PR antagonist compound(s) as the sole active ingredient in the formulation and regimen. The PR antagonist compounds are formulated with a pharmaceutically acceptable carrier or excipient.

Suitably, the PR antagonists used in the invention are formulated for delivery by any suitable route including, e.g., transdermal, mucosal (intranasal, buccal, vaginal), or oral, parenteral, etc, by any suitable delivery device including, e.g., transdermal patches, topical creams or gels, a vaginal ring, among others.

When the compounds are employed for the above utilities, they may be combined with one or more pharmaceutically acceptable carriers or excipients, for example, solvents, diluents and the like. When formulated for oral delivery, the PR antagonist compound can be in the form of a tablet, capsule, caplet, gel tab, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like. When formulated for parenteral delivery, the compositions can be delivered in the form of sterile injectable solutions or suspensions containing from about 0.05 to 5% suspending agent in an isotonic medium. Such pharmaceutical preparations may contain, for example, from about 25 to about 90% of the active ingredient in combination with the carrier, more usually between about 5% and 60% by weight.

The effective dosage of active ingredient employed may vary depending on the particular compound employed, the mode of administration and the severity of the condition being treated. However, in general, satisfactory results are obtained when the compounds of the invention are administered at a daily dosage of from about 0.5 to about 500 mg/kg of animal body weight, about 1 to about 400 mg/kg, about 5 to about 300 mg/kg, about 10 to about 250 mg/kg, about 50 to about 200 mg/kg, or about 100 to 150 mg/kg, desirably given daily or in a sustained release form.

For most large mammals, the total daily dosage is from about 1 to 200 mg, preferably from about 2 to 80 mg. Dosage forms suitable for internal use comprise from about 0.5 to about 500 mg of animal body weight, about 1 to about 400 mg, about 5 to about 300 mg, about 10 to about 250 mg, about 50 to about 200 mg, or about 100 to 150 mg of the active compound in intimate admixture with a pharmaceutically acceptable carrier. This dosage regimen may be adjusted to provide the optimal therapeutic response. For example, several divided doses may be administered daily or the dose may be proportionally reduced as indicated by the exigencies of the therapeutic situation.

These active compounds (one or more PR antagonists) may be administered orally. Solid carriers include starch, lactose, dicalcium phosphate, microcrystalline cellulose, sucrose and kaolin, while liquid carriers include sterile water, non-ionic surfactants, ethanol (e.g., glycerol, propylene glycol and liquid polyethylene glycols), suitable mixtures thereof, and vegetable or edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA.

The preferred pharmaceutical compositions from the standpoint of ease of preparation and administration are solid compositions, particularly tablets and hard-filled or liquid-filled capsules. Oral administration of the compounds is preferred.

These active compounds may also be administered via a vaginal ring. Suitably, use of the vaginal ring is timed to the 28 day cycle. In one embodiment, the ring is inserted into the vagina, and it remains in place for 3 weeks. During the fourth week, the vaginal ring is removed and menses occurs. The following week a new ring is inserted to be worn another 3 weeks until it is time for the next period. In another embodiment, the vaginal ring is inserted weekly, and is replaced for three consecutive weeks. Then, following one week without the ring, a new ring is inserted to begin a new regimen. In yet another embodiment, the vaginal ring is inserted for longer or shorter periods of time.

For use in the vaginal ring, a PR antagonist compound is formulated in a manner similar to that described for contraceptive compounds previously described for delivery via a vaginal ring. See, e.g., U.S. Pat. Nos. 5,972,372; 6,126,958; and 6,125,850.

Optionally, a PR antagonist composition can be formulated for parenteral delivery in a sustained release formulation and administered by injection, e.g., monthly or quarterly.

In another aspect of the invention, an antiprogestin compound is formulated for delivery via a cream or a gel, by a suitable route. Suitably, carriers for such routes are known to those of skill in the art.

In still another aspect of the invention, the PR antagonist compound(s) are delivered via a transdermal patch. Suitably, use of the patch is timed to the 28 day cycle. In one embodiment, the patch is applied via a suitable adhesive on the skin, where it remains in place for 1 week and is replaced weekly for a total period of three weeks. During the fourth week, no patch is applied and menses occurs. The following week a new patch is applied to be worn to begin a new regimen. In yet another embodiment, the patch remains in place for longer, or shorter periods of time.

This invention also includes kits or packages of pharmaceutical formulations designed for use in the regimens described herein. Suitably, the kits contain one or more PR antagonist compounds as described herein.

In one embodiment, the PR antagonist is selected from among mifepristone, onapristone, lilopristone, asoprisinil, CDB-2914, and formulas I and II shown above. In another embodiment, the PR antagonist is selected from among those in U.S. Pat. Nos. 6,391,907; 6,608,086; 6,417,214; 6,380,235; 6,339,098; 6,306,851; 6,369,056; and 6,358,948.

In a further embodiment, the PR antagonist is 3-Chloro-5-(4,4-dimethyl-2-oxo-1,4-dihydro-2H-benzo[d][1,3]oxazin-6-yl)-benzonitrile.

In another embodiment, the PR antagonist is 5-(7-fluoro-3,3-dimethyl-2-oxo-2,3-dihydro-1H-indol-5-yl)-1-methyl-1H-pyrrole-2-carbonitrile.

Advantageously, for use in the kits of the invention, the PR antagonist is formulated for the desired delivery vehicle and route. For example, a PR antagonist can be formulated for oral delivery, parenteral delivery, vaginal ring, transdermal delivery, or mucosal delivery, as discussed in detail above.

In one embodiment, the kit of the invention is designed for daily oral administration over a 28-day cycle, desirably for one oral administration per day, and organized so as to indicate a single oral formulation or combination of oral formulations to be taken on each day of the 28-day cycle. Desirably each kit will include oral tablets to be taken on each the days specified; desirably one oral tablet will contain each of the combined daily dosages indicated. For example, a kit of the invention can contain 21 to 27 daily dosage units of an effective amount of an active agent and, optionally, 1 to 7 daily dosage units of a placebo and other appropriate components including, e.g., instructions for use.

The kit of the invention is preferably a pack (e.g. a blister pack) containing daily doses arranged in the order in which they are to be taken.

In another embodiment, the kit of the invention is designed for weekly or monthly administration via a vaginal ring over a 28-day cycle. Suitably, such a kit contains individual packaging for each of the vaginal rings, i.e. one to three, required for a monthly cycle and other appropriate components, including, e.g., instructions for use.

In another embodiment, the kit of the invention is designed for weekly or monthly administration via a transdermal patch over a 28-day cycle. Suitably, such a kit contains individual packaging for each of the patches, i.e. one to three, required for a monthly cycle and other appropriate components including, e.g., instructions for use.

In still another embodiment, the kit of the invention is designed for parenteral delivery of the PR antagonist. Such a kit is typically designed for delivery at home and may include needles, syringes, and other appropriate packaging and instructions for use.

In yet another embodiment, the kit of the invention contains a PR antagonist compound in a gel or cream formulation. Optionally, the kit can include appropriate packaging such as a tube or other container, an applicator, and/or instructions for use.

In each of the regimens and kits described herein, it is preferred that the daily dosage of each pharmaceutically active component of the regimen remain fixed in each particular phase in which it is administered. It is also understood that the daily dose units described are to be administered in the order described, with the first phase followed in order by the optional second phase. To help facilitate compliance with each regimen, it is also preferred that the kits contain the placebo described for the final days of the cycle. It is further preferred that each package or kit comprise a pharmaceutically acceptable package having indicators for each day of the 28-day cycle, such as a labeled blister package, dial dispenser, or other packages known in the art.

These dosage regimens may be adjusted to provide the optimal therapeutic response. For example, several divided doses of each component may be administered daily or the dose may be proportionally increased or reduced as indicated by the exigencies of the therapeutic situation. In the descriptions herein, reference to a daily dosage unit may also include divided units which are administered over the course of each day of the cycle contemplated.

The following examples are illustrative only and are not intended to be a limitation on the present invention.

### EXAMPLES

### Example 1

### 5-(7-fluoro-3,3-dimethyl-2-oxo-2,3-dihydro-1H-indol-5-yl)-1-methyl-1H-pyrrole-2-carbonitrile

### A. 2,6-difluoronitrobenzene

2,6-Difluoroaniline (11.0 g, 85 mmol) in glacial acetic acid (50 mL) was added slowly to a stirred suspension of sodium perborate tetrahydrate (65 g, 422 mmol) in glacial acetic acid (250 mL) at 80° C. The temperature was maintained between 80-90° C. for 1 hour. The cooled reaction mixture was poured into water and extracted twice with diethylether and the combined organic layers were washed with a dilute solution of sodium bicarbonate, dried (MgSO₄) and evaporated. The residue was purified by silica gel column chromatography (Hexane:THF, 9:1) and the product washed with hexane to afford 2,6-difluoronitrobenzene (7.0 g) which was used without further examination.

### B. 2-(3-Fluoro-2-nitro-phenyl)-malonic acid dimethyl ester

To a solution of 2,6-difluoronitrobenzene (5.0 g, 31.44 mmol) in dry dimethylformamide (DMF −50 mL) was added potassium carbonate (4.41 g, 32 mmol) and dimethylmalonate (3.6 mL, 31.44 mmol). The reaction mixture was heated to 65° C. and stirred for 24 hours. After cooling to room temperature, the mixture was neutralized with dilute aqueous HCl and extracted with diethyl ether, dried (MgSO₄), and concentrated in vacuo. Crystallization from hexane/ethylacetate (95/5), gave 2-(3-fluoro-2-nitro-phenyl)-malonic acid dimethyl ester (4.6 g, 54%). HRMS: calc'd for C₁₁H₁₀FNO₆, 271.0492; found (ESI, [M+H]⁺), 272.0576.

### C. (3-Fluoro-2-nitro-phenyl)-acetic acid

2-(3-Fluoro-2-nitro-phenyl)-malonic acid dimethyl ester (12 g, 44 mmol) in 200 mL 6N hydrochloric acid (6N, 200 mL) was heated under reflux for 4 hours. The mixture was cooled, diluted with 250 mL of water and extracted with diethyl ether, dried (MgSO₄), and concentrated in vacuo. Crystallization from hexane/ethylacetate (95/5) gave (3-fluoro-2-nitro-phenyl)-acetic acid (7.6 g, 54%) which was used without further examination.

### D. 7-Fluoro-1,3-dihydro-indol-2-one

(3-fluoro-2-nitro-phenyl)-acetic acid (9.6 g, 48 mmol) was dissolved in acetic acid (100 mL) and hydrogenated over 10% palladium on carbon (1.3 g) at 50 pounds per square inch (psi) for 24 hours. The catalyst was removed by filtration through the Celite® reagent and the solvent was evaporated. The mixture was then dissolved in ethanol (100 mL), para-toluenesulfonic acid (50 mg) was added and the mixture heated under reflux for 1 hour. The mixture was cooled, poured into water, extracted with ethyl acetate, dried (MgSO₄), and evaporated. The solid was triturated with hexane/ethyl acetate (95/5) to give 7-fluoro-1,3-dihydro-indol-2-one (6 g, 83%). HRMS: calc'd for C₈H₆FNO, 151.0433; found (ESI, [M+H]⁺), 152.0515

### E. 7-Fluoro-3,3-dimethyl-1,3-dihydro-2H-indol-2-one

7-Fluoro-1,3-dihydro-indol-2-one (7.3 g, 48 mmol) and lithium chloride (6.67 g, 158 mmol) was dissolved in THF (200 mL). The solution was then cooled to −78° C. and n-butyllithium (2.5 M, 40 mL, 100 mmol) was added slowly over a 15 minute period. After 20 minutes at −78 ° C., methyl iodide (6 mL, 96 mmol) was added and the mixture allowed to warm to room temperature. After 24 hours, the mixture was poured into water and extracted with ethyl acetate, dried (MgSO₄), and concentrated in vacuo. Flash chromatography (SiO₂, Hexane/ethylacetate 9/1 then 8/2) gave 7-fluoro-3,3-dimethyl-1,3-dihydro-2H-indol-2-one (4.1 g, 48%): HRMS: calc'd for C₁₀H₁₀FNO, 179.0831; found (ESI, [M+H]⁺), 180.0831

### F. 5-Bromo-7-fluoro-3,3-dimethyl-1,3-dihydro-2H-indol-2-one

7-Fluoro-3,3-dimethyl-1,3-dihydro-2H-indol-2-one (4.1 g, 22.9 mmol) was dissolved in dichloromethane (100 mL) and acetic acid (2 mL) at room temperature. Bromine (1.2 mL, 23 mmol) was added and the solution was allowed to stir for 24 hours. The reaction mixture was poured into sodium thiosulfate solution, extracted with diethyl ether, dried (MgSO₄), evaporated and the crude product triturated with hexane to give 5-bromo-7-fluoro-3,3-dimethyl-1,3-dihydro-2H-indol-2-one (4.84 g, 82%): HRMS: calc'd for C₁₀H₉BrFNO, 256.9852; found (ESI, [M−H]⁻), 255.9781.

### G. 5-(7-fluoro-3,3-dimethyl-2-oxo-2,3-dihydro-1H-indol-5-yl)-1-methyl-1H-pyrrole-2-carbonitrile

5-Bromo-7-fluoro-3,3-dimethyl-1,3-dihydro-indol-2-one (5.16 g, 20.0 mmol), 1-methyl-5-cyano-2-pyrroleboronic acid (5.4 g, 36 mmol), KF (3.83 g, 66 mmol), and Pd₂(dba)₃ monochloroform adduct (516 mg, 0.500 mmol) were added to a 200 mL round bottom flask under nitrogen. The flask was sealed and purged with nitrogen for 5 min. THF (50 mL) was added and the mixture was purged with nitrogen for an additional 5 min. A solution of tri-t-butylphosphine (10% wt in hexanes) (2.97 mL, 1.00 mmol) was added via syringe and the mixture was stirred vigorously at 25° C. for 5 hours. The mixture was diluted with 250 mL of EtOAc, filtered through a plug of silica gel, washed through with 200 mL of EtOAc and concentrated to give a crude brown/black semi-solid. Purification by flash chromatography (20% acetone/hexane) afforded the title compound (4.5 g, 80%) as an off-white solid. HRMS: calc'd for C₁₆H₁₄FN₃O, 283.1121; found (ESI, [M−H]⁻), 282.1034

Analytical HPLC: Major=98.9% at 210-370 nm window=99.2% at 286 nm (max. abs) RT=8.7 min, 85/15-5/95 (Ammon. Form. Buff. pH=3.5/ACN+MeOH) for 10 min, hold 4 min, the Xterra® instrument RP18, 3.5μ, 150×4.6 mm.

### Example 2

### 5-(4-fluoro-3,3-dimethyl-2-oxo-2,3-dihydro-1H-indol-5-yl)-1-methyl-1H-pyrrole-2-carbonitrile

### A. 2-(2-Fluoro-6-nitro-phenyl)-malonic acid dimethyl ester

To a solution of 2,3-difluoronitrobenzene (9 g, 56 mmol) in DMF was added potassium carbonate (13.8 g, 100 mmol) and dimethylmalonate (6.88 mL, 60 mmol). The reaction mixture was heated to 65° C. and stirred 24 hours. The mixture was cooled, neutralized with dilute HCl and extracted with diethyl ether, the organic layers were dried over magnesium sulfate, and concentrated in vacuo. The crude product was recrystallized from hexane/ethylacetate (95/5), and filtered to afford 2-(2-fluoro-6-nitro-phenyl)-malonic acid dimethyl ester (6.6 g, 43%).

### B. (2-fluoro-6-nitrophenyl)acetic acid

2-(2-Fluoro-6-nitro-phenyl)-malonic acid dimethyl ester (6.5 g, 23.98 mmol) was refluxed in 200 mL 6N hydrochloric acid for 24 hours. The solid was collected by suction filtration, and dried to give 3.3 g, 54% yield of the title compound.

### C. 4-fluoro-1,3-dihydro-2H-indol-2-one

(2-fluoro-6-nitrophenyl)acetic acid (3.3 g, 16.6 mmol) was dissolved in acetic acid (20 mL) and hydrogenated over palladium on carbon (10%, 0.5 g) at 50 psi for 24 hours. The catalyst was removed by filtration through the Celite® reagent, which was washed with methanol, and the combined organics were then evaporated. The reaction mixture was then dissolved in ethanol (100 mL), 50 mg of para-toluenesulfonic acid was added, and the mixture heated under reflux for 1 hour. The mixture was poured into water, extracted with ethyl acetate, dried over magnesium sulfate, and evaporated. The solid was triturated with hexane/ethyl acetate (95/5) to give 1.7 g, 67% of 4-fluoro-1,3-dihydro-2H-indol-2-one: HRMS [M+H]⁺=152.0515

### D. 4-Fluoro-3,3-dimethyl-1,3-dihydro-2H-indol-2-one

4-Fluoro-1,3-dihydro-2H-indol-2-one (3.4 g, 22.5 mmol) and lithium chloride (2.7 g, 60 mmol) was dissolved in THF (100 mL). The solution was then cooled to −78° C. and n-butyllithium (7 mL, 2.5M in hexanes, 15 mmol) was added slowly over a 15 minute period. Methyl iodide (3.08 mL, 50 mmol) was added and the mixture was allowed to warm up to room temperature. After 24 hours, the mixture was poured into water and extracted with ethyl acetate, dried over magnesium sulfate, and concentrated in vacuo. Flash chromatography (SiO₂, Hexane/ethylacetate 9/1 then 8/2) gave 4-fluoro-3,3-dimethyl-1,3-dihydro-2H-indol-2-one (1.0 g, 25%)

### E. 5-Bromo-4-fluoro-3,3-dimethyl-1,3-dihydro-2H-indol-2-one

4-Fluoro-3,3-dimethyl-1,3-dihydro-2H-indol-2-one (1 g, 22.9 mmol) was dissolved in dichloromethane (DCM) (50 mL) and acetic acid (2 mL) at room temperature. Bromine (0.386 mL, 7.5 mmol) was added and the solution was allowed to stir 24 hours. The reaction mixture was poured into sodium thiosulfate solution, extracted with diethylether, the combined organic layers were dried over magnesium sulfate and evaporated. Trituration of the crude product with hexane gave 5-bromo-4-fluoro-3,3-dimethyl-1,3-dihydro-2H-indol-2-one (1.25 g, 87%): HRMS [M−H]⁻ 255.9781

### F. 5-(4-fluoro-3,3-dimethyl-2-oxo-2,3-dihydro-1H-indol-5-yl)-1-methyl-1H-pyrrole-2-carbonitrile

5-bromo-4-fluoro-3,3-dimethyl-1,3-dihydro-2H-indol-2-one (1.25 g, 4.86 mmol) and tetrakis(triphenylphosphine)palladium(0) (0.4 g) were dissolved in ethylene glycol dimethyl ether (40 mL) and stirred 15 minutes. N-methyl-5-cyanopyrroleboronic acid (2.0 g, 13.33 mmol) and potassium carbonate (3.48, 25 mmol) were added followed by water (20 mL) and the mixture heated under reflux (24 hours). The mixture was then poured into water, neutralized with dilute hydrochloric acid, and extracted with ethylacetate. The solvent was dried over magnesium sulfate, and concentrated in vacuo. Flash chromatography; SiO₂, Hexane/THF 9/1 then 7/3 gave 5-(4-fluoro-3,3-dimethyl-2-oxo-2,3-dihydro-1H-indol-5-yl)-1-methyl-1H-pyrrole-2-carbonitrile (0.060 g, 5%): HRMS: calcd for C₁₆H₁₄FN₃O, 283.1121; found (ESI, [M+H]⁺), 284.1121.

Analytical HPLC: Retention time=8.8 min, purity=100% at 210-300 nm and 100% at 274 nm (max absorption), 85/15-5/95 (Ammon. Form. Buff. pH=3.5/ACN+MeOH) for 10 min, hold 4 min, the Xterra® RP18 instrument, 3.5μ, 150×4.6 mm.

### Example 3

### 5-(7′-fluoro-2′-oxo-1′,2′-dihydrospiro[cyclopropane-1,3′-indol]-5′-yl)-1-methyl-1H-pyrrole-2-carbonitrile

### A. 7′-fluorospiro[cyclopropane-1,3′-indol]-2′(1′H) -one

7-Fluorooxindole (1.28 g, 8.50 mmol) and lithium chloride (0.899 g, 21.3 mmol) were suspended in 80 mL of THF and cooled to 0° C. n-Butyllithium (8.5 mL, 16.9 mmol) was added slowly, the mixture was stirred for 20 minutes, and then dibromoethane (0.73 mL, 8.5 mmol) was added. The mixture was warmed to 25° C. and stirred for 16 hours. The reaction was quenched with saturated aqueous NH₄Cl and diluted with ether. The organics were washed with water, brine, dried over MgSO₄, and concentrated. Flash chromatography (10% acetone/hexane) afforded 0.54 g (36%) of 7′-fluorospiro[cyclopropane-1,3′-indol]-2′(1′H)-one as a white solid: HRMS: calcd for C₁₀H₈FNO, 177.0590; found (ESI, [M+H]⁺), 178.0659

Analytical HPLC: retention time 6.6 min, 210-370 nm, the Xterra® RP18 instrument, 3.5μ, 150×4.6 mm 40 C 85/15-5/95 (Ammon. Form. Buff. pH=3.5/ACN+MeOH) for 10 min, hold 4 min 1.2 mL/min 5 μL injection.

B. 5′-Bromo-7′-fluorospiro[cyclopropane-1,3′-indol]-2′(1′H)-one

7′-Fluorospiro[cyclopropane-1,3′-indol]-2′(1′H)-one (0.54 g, 3.05 mmol) was dissolved in 20 mL of CH₂Cl₂ and sodium acetate (0.28 g, 3.36 mmol) was added followed by bromine (0.173 mL, 3.36 mmol). The mixture was stirred at 25° C. for 16 hours then diluted with ether and washed with Na₂S₃O₃, sodium bicarbonate, water, brine, dried over MgSO₄, and concentrated. Purification by flash chromatography (15% acetone/hexane) afforded 5′-Bromo-7′-fluorospiro[cyclopropane-1,3-indol]-2′(1′H)-one (0.64 g, 82%) as a white solid

Analytical HPLC: retention time 8.4 min, 210-370 nm the Xterra® RP18 instrument, 3.5μ, 150×4.6 mm 40 C 85/15-5/95 (Ammon. Form. Buff. pH=3.5/ACN+MeOH) for 10 min, hold 4 min 1.2 mL/min 5μL injection.

### C. 5-(7′-fluoro-2′-oxo-1′,2′-dihydrospiro[cyclopropane-1,3′-indol]-5′-yl)-1-methyl-1H-pyrrole-2-carbonitrile

5′-Bromo-7′-fluorospiro[cyclopropane-1,3′-indol]-2′(1′H)-one (0.60 g, 2.3 mmol), 1-methyl-5-cyano-2-pyrroleboronic acid (0.63 g, 4.2 mmol), KF (0.44 g, 7.6 mmol), and Pd₂(dba)₃ monochloroform adduct (60 mg, 0.058 mmol) were added to a vial and then purged with nitrogen. THF (5.5 mL) was added and the mixture was purged with nitrogen for 5 minutes. A solution of tri-t-butylphosphine (10% wt in hexanes) (0.342 mL, 0.115 mmol) was added via syringe and the mixture was stirred vigorously at 25° C. for 2.5 hours. The mixture was diluted with 100 mL of EtOAc and filtered through a plug of silica gel and concentrated. Purification by flash chromatography (25% acetone/hexane) afforded the title compound (0.53 g, 83%) as a white solid. MP 228-231° C.

Analytical HPLC: retention time 8.6 min, 210-370 nm, the Xterra® RP18 instrument, 3.5μ, 150×4.6 mm 40° C. 85/15-5/95 (Ammon. Form. Buff. pH=3.5/ACN+MeOH) for 10 min, hold 4 min 1.2 mL/min 5 μL injection.

### Example 4

### 5-(7-fluoro-1,3,3-trimethyl-2-oxo-2,3-dihydro-1H-indol-5-yl)-1-methyl-1H-pyrrole-2-carbonitrile

### A. 7-fluoro-1,3,3-trimethyl-1,3-dihydro-2H-indol-2-one

7-Fluorooxindole (1.51 g, 10 mmol) and lithium chloride (1.06 g, 25 mmol) were suspended in 30 mL of THF and cooled to 0° C. n-Butyllithium (10 mL, 20 mmol) was added slowly and the mixture was stirred for 20 minutes. Iodomethane (1.24 mL, 20 mmol) was added and the mixture was stirred at 0° C. for 1 hour then warmed to 25° C. and stirred for 16 hours. The reaction was quenched with saturated aqueous NH₄Cl and diluted with ethyl acetate. The organics were washed with water, saturated aqueous NaCl, dried over MgSO₄, and concentrated. Flash chromatography (5% acetone/hexane) afforded the title compound 0.12 g (7%) as a white solid.

HRMS: calcd for C₁₁H₁₂FNO, 193.0903; found (ESI, [M+H]⁺), 194.0976;

### B. 5-bromo-7-fluoro-1,3,3-trimethyl-1,3-dihydro-2H-indol-2-one

7-Fluoro-1,3,3-trimethyl-1,3-dihydro-2H-indol-2-one (0.10 g, 0.52 mmol) was dissolved in 5 mL of CH₂Cl₂ and sodium acetate (47 mg, 0.56 mmol) was added followed by bromine (0.029 mL, 0.56 mmol). The mixture was stirred at 25° C. for 16 hours then loaded directly onto a silica gel column. The column was eluted with 250 mL of CH₂Cl₂ and 250 mL 5% Acetone/CH₂Cl₂ to provide the title compound (116 mg) as a white solid (82%).

HRMS: calcd for C₁₁H₁₁BrFNO, 271.0008; found (ESI, [M+H]⁺), 272.0088;

Analytical HPLC: retention time 9.4 min, 210-370 nm, the Xterra® RP18 instrument, 3.5μ, 150×4.6 mm 40° C. 85/15-5/95 (Ammon. Form. Buff. pH=3.5/ACN+MeOH) for 10 min, hold 4 min 1.2 mL/min 5 μL injection.

### C. 5-(7-fluoro-1,3,3-trimethyl-2-oxo-2,3-dihydro-1H-indol-5-yl)-1-methyl-1H-pyrrole-2-carbonitrile

5-Bromo-7-fluoro-1,3,3-trimethyl-1,3-dihydro-2H-indol-2-one (0.10 g, 0.36 mmol), 1-methyl-5-cyano-2-pyrroleboronic acid (95 mg, 0.63 mmol), and KF (69 mg, 1.19 mmol) were suspended in 1 mL of dioxane. Pd₂(dba)₃ monochloroform adduct (3.1 mg, 0.003 mmol) and Pd(P(t-Bu)₃)₂ (4.6 mg, 0.009 mmol) were added and the mixture was stirred vigorously at 45° C. for 6 hours. The mixture was diluted with 100 mL of EtOAc and filtered through a plug of silica gel and concentrated. Purification by flash chromatography (2% acetone/hexane) afforded the title compound (30 mg, 28%) as a tan solid.

HRMS: calcd for C₁₇H₁₆FN₃O, 297.1277; found (ESI, [M+H]⁺), 298.1366;

Analytical HPLC: retention time 9.4 min, 210-370 nm, the Xterra® RP18 instrument, 3.5μ, 150×4.6 mm 40° C. 85/15-5/95 (Ammon. Form. Buff. pH=3.5/ACN+MeOH) for 10 min, hold 4 min 1.2 mL/min 5 μL injection.

### Example 5

### 5-(7-fluoro-3,3-dimethyl-2-oxo-2,3-dihydro-1H-indol-5-yl)-1H-pyrrole-2-carbonitrile

### A. tert-butyl 2-(7-fluoro-3,3-dimethyl-2-oxo-2,3-dihydro-1H-indol-5-yl)-1H-pyrrole-1-carboxylate

A vial was charged with 5-bromo-7-fluoro-3,3-dimethyl-1,3-dihydro-2H-indol-2-one (1.0 g, 3.5 mmol), 1-tert-butoxycarbonyl-2-pyrroleboronic acid (1.12 g, 5.3 mmol), KF (0.67 g, 11.5 mmol), and Pd₂(dba)₃ monochloroform adduct (54 mg, 0.053 mmol) and placed under a nitrogen atmosphere. THF (8 mL) was added and the mixture was purged with nitrogen for 5 minutes. P(t-Bu)₃ (10% wt. solution in hexane 0.370 mL, 0.126 mmol) was added via syringe and the mixture was stirred at 25° C. for 16 hours. The mixture was diluted with EtOAc and filtered through a plug of silica gel and concentrated. Purification by flash chromatography (500 mL 25% hexane/CH₂Cl₂, then 500 mL 100% CH₂Cl₂, then 500 mL 5% ethyl acetate/CH₂Cl₂) afforded the title compound (1.06 g, 88%) as colorless crystals.

HRMS: calcd for C₁₉H₂₁FN₂O₃+H, 345.16145; found (ESI, [M+H]⁺), 345.1629.

Analytical HPLC: retention time 10.0 min, 210-370 nm, the Xterra® RP18 instrument, 3.5μ, 150×4.6 mm 40 C 85/15-5/95 (Ammon. Form. Buff. pH=3.5/ACN+MeOH) for 10 min, hold 4 min 1.2 mL/min 5 μL injection.

### B. tert-butyl 2-cyano-5-(7-fluoro-3,3-dimethyl-2-oxo-2,3-dihydro-1H-indol-5-yl)-1 H-pyrrole-1-carboxylate

To a stirred solution of tert-butyl 2-(7-fluoro-3,3-dimethyl-2-oxo-2,3-dihydro-1H-indol-5-yl)-1H-pyrrole-1-carboxylate (1.0 g, 2.9 mmol) was added chlorosulfonylisocyanate (0.28 mL, 3.2 mmol). The mixture was stirred at 25° C. for 2 hours, then DMF (0.21 mL, 2.9 mmol) was added and the mixture was stirred for an additional 1 hour. The mixture was diluted with ethyl acetate and washed with NaHCO₃, water, saturated aqueous NaCl, dried over MgSO₄, and concentrated. Flash chromatography (2% MeOH/CH₂Cl₂) afforded 0.23 g (21%) of the title compound as a white solid.

HRMS: calcd for C₂₀H₂₀FN₃O₃+H, 370.15670; found (ESI, [M+H]⁺), 370.1554.

Analytical HPLC: retention time 9.5 min, 210-370 nm, the Xterra® RP18 instrument, 3.5μ, 150×4.6 mm 40° C. 85/15-5/95 (Ammon. Form. Buff. pH=3.5/ACN+MeOH) for 10 min, hold 4 min 1.2 mL/min 5 μL injection.

### C. 5-(7-fluoro-3,3-dimethyl-2-oxo-2,3-dihydro-1H-indol-5-yl)-1H-pyrrole-2-carbonitrile

tert-butyl 2-cyano-5-(7-fluoro-3,3-dimethyl-2-oxo-2,3-dihydro-1H-indol-5-yl)-1H-pyrrole-1-carboxylate (0.18 g, 0.50 mmol) was dissolved in 10 mL of dimethylacetamide and the solution was heated to 180° C. for 1 hour. The mixture was cooled, diluted with ethyl acetate and washed with water, saturated aqueous NaCl, dried over MgSO₄, and concentrated. Flash chromatography (25% acetone/hexane) afforded 0.121 g (91%) of the title compound as a white solid.

HRMS: calcd for C₁₅H₁₂FN₃O+H, 270.10426; found (ESI, [M+H]⁺), 270.1053.

Analytical HPLC: retention time 8.7 min, 210-370 nm, the Xterra® RP18 instrument, 3.5μ, 150×4.6 mm 40 C 85/15-5/95 (Ammon. Form. Buff. pH=3.5/ACN+MeOH) for 10 min, hold 4 min 1.2 mL/min 5 μL injection.

### Example 6

### General Procedure for Alkylation of 5-(7-fluoro-3,3-dimethyl-2-oxo-2,3-dihydro-1H-indol-5-yl)-1-methyl-1H-pyrrole-2-carbonitrile

To a solution of 5-(7-fluoro-3,3-dimethyl-2-oxo-2,3-dihydro-1H-indol-5-yl)-1-methyl-1H-pyrrole-2-carbonitrile (0.10 g, 0.35 mmol) in dry THF (2 mL) was added potassium tert-butoxide (1 M solution in THF, 1 mL, 1 mmol). The mixture was stirred at room temperature for 1 hour. After this time, the appropriate alkylating agent (alkyl iodide or alkyl bromide) (0.5 mmol) was added by syringe. The resultant mixture was stirred overnight, then evaporated and subjected to purification by silica gel column chromatography (EtOAc/Hexane, gradient elution).

The compounds were characterized by high resolution mass spectrometry and HPLC. The HPLC conditions used were: the Xterra®V RP18 column, 3.5μ, 150×4.6 mm, Flow Rate 1.2 mL/min, Mobile Phase Comp. 85/15-5/95 (Ammon. Form. Buff. pH=3.5/ACN+MeOH); Detection: 210-370 nm

The following compounds were prepared by this procedure:

### A. methyl[5-(5-cyano-1-methyl-1H-pyrrol-2-yl)-7-fluoro-3,3-dimethyl-2-oxo-2,3-dihydro-1H-indol-1-yl]acetate

Quantity obtained: 0.087 g

Alkylating agent: methyl bromoacetate (0.047 mL)

Analytical HPLC purity: 99.7%

Analytical HPLC retention time: 9.2 minutes

HRMS: calcd for C₁₉H₁₈FN₃O₃+H, 356.14050; found (ESI, [M+H]⁺), 356.142

### B. 5-(1-ethyl-7-fluoro-3,3-dimethyl-2-oxo-2,3-dihydro-1H-indol-5-yl)-1-methyl-1H-pyrrole-2-carbonitrile

Quantity obtained: 0.0723 g

Alkylating agent: ethyl iodide (0.040 mL)

Analytical HPLC purity: 99.9%

Analytical HPLC retention time: 9.8 minutes.

HRMS: calcd for C₁₈H₁₈FN₃O+H, 312.15067; found (ESI, [M+H]⁺), 312.1524; (delta 32 6 ppm)

### C. 5-(7-fluoro-3,3-dimethyl-2-oxo-1-prop-2-yn-1-yl-2,3-dihydro-1H-indol-5-yl)-1-methyl-1H-pyrrole-2-carbonitrile

Quantity obtained: 0.050 g

Alkylating agent: propargyl bromide (0.045 mL)

Analytical HPLC purity: 95.9%

Analytical HPLC retention time: 9.5 minutes

HRMS: calcd for C₁₉H₁₆FN₃O+H, 322.13502; found (ESI, [M+H]⁺), 322.135

### D. 5-[7-fluoro-3,3-dimethyl-2-oxo-1-(2-phenylethyl)-2,3-dihydro-1H-indol-5-yl]-1-methyl-1H-pyrrole-2-carbonitrile

Quantity obtained: 0.041 g

Alkylating agent: phenethyl bromide (0.067 mL)

Analytical HPLC purity: 100%

Analytical HPLC retention time: 10.8 minutes

HRMS: calcd for C₂₄H₂₂FN₃O+H, 388.18197; found (ESI, [M+H]⁺), 388.1806

### E. 5-(1-benzyl-7-fluoro-3,3-dimethyl-2-oxo-2,3-dihydro-1H-indol-5-yl)-1-methyl-1H-pyrrole-2-carbonitrile

Quantity obtained: 0.0766 g

Alkylating agent: benzyl bromide (0.059 mL)

Analytical HPLC purity: 100%

Analytical HPLC retention time: 10.5 minutes

HRMS: calcd for C₂₃H₂₀FN₃O+H, 374.16632; found (ESI, [M+H]⁺), 374.1685; (delta=6 ppm)

### F. 5-(7-fluoro-3,3-dimethyl-2-oxo-1-propyl-2,3-dihydro-1H-indol-5-yl)-1-methyl-1H-pyrrole-2-carbonitrile

Quantity obtained: 0.070 g

Alkylating agent: iodo propane (0.049 mL)

Analytical HPLC purity: 100%

Analytical HPLC retention time: 10.3 minutes

HRMS: calcd for C₁₉H₂₀FN₃O+H, 326.16632; found (ESI, [M+H]⁺), 326.1652

### G. 5-(7-fluoro-1-isobutyl-3,3-dimethyl-2-oxo-2,3-dihydro-1H-indol-5-yl)-1-methyl-1H-pyrrole-2-carbonitrile

Quantity obtained: 0.0662 g

Alkylating agent: 2-methyliodopropane (0.060 mL)

Analytical HPLC purity: 100%

Analytical HPLC retention time: 10.6 minutes

HRMS: calcd for C₂₀H₂₂FN₃O+H, 340.18197; found (ESI, [M+H]⁺), 340.1838

### H. 5-(7-fluoro-1-isopropyl-3,3-dimethyl-2-oxo-2,3-dihydro-1H-indol-5-yl)-1-methyl-1H-pyrrole-2-carbonitrile

Quantity obtained: 0.055 g

Alkylating agent: isopropyl iodide (0.049 mL)

Analytical HPLC purity: 98.8%

Analytical HPLC retention time: 10.3 minutes

HRMS: calcd for C₁₉H₂₀FN₃O+H, 326.16632; found (ESI, [M+H]⁺), 326.1661

### I. 5-(1-allyl-7-fluoro-3,3-dimethyl-2-oxo-2,3-dihydro-1H-indol-5-yl)-1-methyl-1H-pyrrole-2-carbonitrile

Quantity obtained: 0.077 g

Alkylating agent: allyl iodide (0.045 mL)

Analytical HPLC purity: 99.6%

Analytical HPLC retention time: 9.9 minutes

HRMS: calcd for C₁₉H₁₈FN₃O+H, 324.15067; found (ESI, [M+H]⁺), 324.1512

### J. 5-(1-cyclohexyl-7-fluoro-3,3-dimethyl-2-oxo-2,3-dihydro-1H-indol-5-yl)-1-methyl-1H-pyrrole-2-carbonitrile

Quantity obtained: 0.0037 g

Alkylating agent: cyclohexyl iodide (0.064 mL)

Analytical HPLC purity: 94.3%,

Analytical HPLC retention time: 11.2 minutes

HRMS: calcd for C₂₂H₂₄FN₃O+H, 366.19762; found (ESI, [M+H]⁺), 366.1978

### K. 5-(1-cyclopentyl-7-fluoro-3,3-dimethyl-2-oxo-2,3-dihydro-1H-indol-5-yl)-1-methyl-1H-pyrrole-2-carbonitrile

Quantity obtained: 0.034 g

Alkylating agent: cyclopentyl iodide (0.057 mL)

Analytical HPLC purity: 100%

Analytical HPLC retention time: 10.9 minutes.

HRMS: calcd for C₂₁H₂₂FN₃O+H, 352.18197; found (ESI, [M+H]⁺), 352.184; (delta=6 ppm).

### Example 7

Cyclic Regimen Using PR Antagonists

A phase 2, randomized, double-blind, multicenter, dose-ranging study of 3 doses of each of the compounds of Table 1 in a 21-day regimen followed by 7 days of placebo pills, and a comparator (the combination steroidal OC desogestrel (DSG) 150 μg/20 μg ethinyl estradiol for 21 days followed by 2 days of placebo pills, followed by 5 days of 10 μg EE, marketed in the United States under the name Mircette) is planned.

TABLE 1

| Example | Compound |
|---|---|
| 1 | 5-(7-fluoro-3,3-dimethyl-2-oxo-2,3-dihydro-1H-indol-5-yl)- |
| | 1-methyl-1H-pyrrole-2-carbonitrile |
| 3 | 5-(4-fluoro-3,3-dimethyl-2-oxo-2,3-dihydro-1H-indol-5-yl)- |
| | 1-methyl-1H-pyrrole-2-carbonitrile |
| 5 | 5-(7′-fluoro-2′-oxo-1′,2′-dihydrospiro[cyclopropane-1,3′-indol]- |
| | 5′-yl)-1-methyl-1H-pyrrole-2-carbonitrile |
| 8 | 5-(7-fluoro-2-oxo-1,3,3-trimethyl-2-oxo-2,3-dihydro-1H- |
| | indol-5-yl)-1-methyl-1H-pyrrole-2-carbonitrile |
| 9 | 5-(7-fluoro-3,3-dimethyl-2-oxo-2,3-dihydro-1H-indol-5-yl)- |
| | 1H-pyrrole-2-carbonitrile |
| 9B | tert-butyl-2-cyano-5-(7-fluoro-3,3-dimethyl-2-oxo-2,3- |
| | dihydro-1H-indol-5-yl)-1H-pyrrole-1-carboxylate |
| 10A | Methyl-[5-(5-cyano-1-methyl-1H-pyrrol-2-yl)-7-fluoro- |
| | 3,3-dimethyl-2-oxo-2,3-dihydro-1H-indol-1-yl]acetate |
| 10B | 5-(1-ethyl-7-fluoro-3,3-dimethyl-2-oxo-2,3-dihydro-1H-indol- |
| | 5-yl)-1-methyl-1H-pyrrole-2-carbonitrile |
| 10C | 5-(7-fluoro-3,3-dimethyl-2-oxo-1-prop-2-yn-1-yl-2,3-dihydro- |
| | 1H-indol-5-yl)-1-methyl-1H-pyrrole-2-carbonitrile |
| 10D | 5-[7-fluoro-3,3-dimethyl-2-oxo-1(2-phenylethyl)-2,3-dihydro- |
| | 1H-indol-5-yl]-1-methyl-1H-pyrrole-2-carbonitrile |
| 10E | 5-(1-benzyl-7-fluoro-3,3-dimethyl-2-oxo-2,3-dihydro-1H- |
| | indol-5-yl)-1-methyl-1H-pyrrole-2-carbonitrile |
| 10F | 5-(7-fluoro-3,3-dimethyl-2-oxo-1-propyl-2,3-dihydro-1H- |
| | indol-5-yl)-1-methyl-1H-pyrrole-2-carbonitrile |
| 10G | 5-(7-fluoro-1-isobutyl-3,3-dimethyl-2-oxo-2,3-dihydro-1H- |
| | indol-5-yl)-1-methyl-1H-pyrrole-2-carbonitrile |
| 10H | 5-(7-fluoro-1-isopropyl-3,3-dimethyl-2-oxo-2,3-dihydro-1H- |
| | indol-5-yl)-1-methyl-1H-pyrrole-2-carbonitrile |
| 10I | 5-(1-allyl-7-fluoro-3,3-dimethyl-2-oxo-2,3-dihydro-1H-indol- |
| | 5-yl)-1-methyl-1H-pyrrole-2-carbonitrile |
| 10J | 5-(1-cyclohexyl-7-fluoro-3,3-dimethyl-2-oxo-2,3-dihydro-1H- |
| | indol-5-yl)-1-methyl-1H-pyrrole-2-carbonitrile |
| 10K | 5-(1-cyclopentyl-7-fluoro-3,3-dimethyl-2-oxo-2,3-dihydro-1H- |
| | indol-5-yl)-l-methyl-1H-pyrrole-2-carbonitrile |

Approximately 20 sites will participate with approximately 16 subjects per site.

The study will have 2 parts. Part 1 (days 1-84) of the study will evaluate the ability of the compounds of Table 2 to produce ovarian suppression, along with evaluating cycle control, side effects, and metabolic data. Part 2 (days 85-168) will continue to follow the subjects to collect cycle control, side effects, and metabolic data. Each subject will participate for up to 9 months, depending on the length of the subject's screening period. Eight (8) cycles will be observed. The first cycle will be a baseline observation of ovulation. Six (6) treatment cycles will be followed by 1 post-treatment observation cycle to assess return to ovulation. The investigator will have approximately 9 months to enroll subjects.

The subjects will be healthy women of ≧18 years of age who are younger than 36 years at the time of randomization. Subjects must have had spontaneous regular (24- to 32-day) menstrual cycles for the 3-month period preceding entry into the pretreatment observation cycle, excluding postabortal and nonbreastfeeding postpartum subjects. The pretreatment observation cycle for all subjects will begin on day 1 of the subsequent spontaneous menses after completion of the prestudy screening (visit 1).

The pretreatment observation cycle is a control cycle; no test article will be administered. Each subject will begin test article on the first day of her menstrual bleeding (first subject pack only). Each subject pack will contain a compound of Table 2 or the steroid combination OC comparator. Subjects will take 3-Chloro-5-(4,4-dimethyl-2-oxo-1,4-dihydro-2H-benzo[d][1,3]oxazin-6-yl)-benzonitrile orally, once daily for 21 days (days 1 through 21), followed by 7 days of placebo pills (days 22 through 28) for 6 cycles. Subjects assigned to a steroid combination OC comparator, DSG 150 μg, will take test article orally, once daily for 21 days (days 1 through 21), followed by 2 days of placebo pills (days 22 through 23), followed by 5 days of 10 μg EE (days 24 through 28) for 6 cycles. There will also be a post-treatment cycle in which no test article will be administered and return to ovulation will be assessed.

Each subject will be randomly assigned to receive one of the following:

| Group | Treatment |
|---|---|
| A | 10 mg of a compound of Table 2 for 21 days followed by 7 |
| | days of placebo pills |
| B | 20 mg of a compound of Table 2 for 21 days followed by 7 |
| | days of placebo pills |
| C | 30 mg of a compound of Table 2 for 21 days followed by 7 |
| | days of placebo pills |
| D | Desogestrel 150 μg for 21 days followed by 2 days of placebo |
| | pills, followed by 5 days of 10 μg EE |

Each subject will begin the test article on the first day of her menstrual bleeding (first subject pack only). Subjects will take test article orally, once daily for 28 days, at approximately the same time each day. All subsequent subject packs will begin following day 28 of the previous pill pack. Subjects will take test article daily without interruption during the treatment cycles.

It is anticipated that one or more treatment groups A, B and C receiving a regimen of the invention will have experience effective contraception, inhibition of ovulation, and all groups will have menses during the fourth week of each month of treatment.

### Example 8

### Cyclic Regimen Using the 3-Chloro-5-(4,4-dimethyl-2-oxo-1,4-dihydro-2H-benzo[d][1,3]oxazin-6-yl)-benzonitrile PR Antagonist Compound

A phase 2, randomized, double-blind, multicenter, dose-ranging study of 3 doses of 3-Chloro-5-(4,4-dimethyl-2-oxo-1,4-dihydro-2H-benzo[d][1,3]oxazin-6-yl)-benzonitrile in a 21-day regimen followed by 7 days of placebo pills, and a comparator (the combination steroidal OC desogestrel (DSG) 150 μg/20 μg ethinyl estradiol for 21 days followed by 2 days of placebo pills, followed by 5 days of 10 μg EE, marketed in the United States under the name Mircette) is planned.

Approximately 20 sites will participate with approximately 16 subjects per site.

The study will have 2 parts. Part 1 (days 1-84) of the study will evaluate the ability of 3-Chloro-5-(4,4-dimethyl-2-oxo-1,4-dihydro-2H-benzo[d][1,3]oxazin-6-yl)-benzonitrile to produce ovarian suppression, along with evaluating cycle control, side effects, and metabolic data. Part 2 (days 85-168) will continue to follow the subjects to collect cycle control, side effects, and metabolic data. Each subject will participate for up to 9 months, depending on the length of the subject's screening period. Eight (8) cycles will be observed. The first cycle will be a baseline observation of ovulation. Six (6) treatment cycles will be followed by 1 posttreatment observation cycle to assess return to ovulation. The investigator will have approximately 9 months to enroll subjects.

The subjects will be healthy women of ≧18 years of age who are younger than 36 years at the time of randomization. Subjects must have had spontaneous regular (24- to 32-day) menstrual cycles for the 3-month period preceding entry into the pretreatment observation cycle, excluding postabortal and nonbreastfeeding postpartum subjects. The pretreatment observation cycle for all subjects will begin on day 1 of the subsequent spontaneous menses after completion of the prestudy screening (visit 1).

The pretreatment observation cycle is a control cycle; no test article will be administered. Each subject will begin test article on the first day of her menstrual bleeding (first subject pack only). Each subject pack will contain 3-Chloro-5-(4,4-dimethyl-2-oxo-1,4-dihydro-2H-benzo[d][1,3]oxazin-6-yl)-benzonitrile or the steroid combination OC comparator. Subjects will take 3-Chloro-5-(4,4-dimethyl-2-oxo-1,4-dihydro-2H-benzo[d][1,3]oxazin-6-yl)-benzonitrile orally, once daily for 21 days (days 1 through 21), followed by 7 days of placebo pills (days 22 through 28) for 6 cycles. Subjects assigned to a steroid combination OC comparator, DSG 150 μg, will take test article orally, once daily for 21 days (days 1 through 21), followed by 2 days of placebo pills (days 22 through 23), followed by 5 days of 10 μg EE (days 24 through 28) for 6 cycles. There will also be a posttreatment cycle in which no test article will be administered and return to ovulation will be assessed.

Each subject will be randomly assigned to receive one of the following:

| Group | Treatment |
|---|---|
| A | 10 mg of 3-Chloro-5-(4,4-dimethyl-2-oxo-1,4-dihydro-2H- |
| | benzo[d][1,3]oxazin-6-yl)-benzonitrile for 21 days followed by |
| | 7 days of placebo pills |
| B | 20 mg of 3-Chloro-5-(4,4-dimethyl-2-oxo-1,4-dihydro-2H- |
| | benzo[d][1,3]oxazin-6-yl)-benzonitrile for 21 days followed by |
| | 7 days of placebo pills |
| C | 30 mg of 3-Chloro-5-(4,4-dimethyl-2-oxo-1,4-dihydro-2H- |
| | benzo[d][1,3]oxazin-6-yl)-benzonitrile for 21 days followed by |
| | 7 days of placebo pills |
| D | Desogestrel 150 μg for 21 days followed by 2 days of placebo |
| | pills, followed by 5 days of 10 μg EE |

Each subject will begin test article on the first day of her menstrual bleeding (first subject pack only). Subjects will take test article orally, once daily for 28 days, at approximately the same time each day. All subsequent subject packs will begin following day 28 of the previous pill pack. Subjects will take test article daily without interruption during the treatment cycles.

It is anticipated that one or more treatment groups A, B and C receiving a regimen of the invention will have experience effective contraception, inhibition of ovulation, and all groups will have menses during the fourth week of each month of treatment.

### Example 9

### Kit of the Invention

A blister pack with 28 blister containers is made with a cardboard, paperboard, foil or plastic backing and enclosed in a suitable cover. The blister containers are arranged to house a sequence of 21 pills each providing a daily dose of 10 mg of 3-Chloro-5-(4,4-dimethyl-2-oxo-1,4-dihydro-2H-benzo[d][1,3]oxazin-6-yl)-benzonitrile followed by 7 daily doses of placebo pills (or 7 empty blisters). Each blister container may conveniently be numbered or otherwise marked, e.g. starting with the first of the 21 dosage units that contain the active ingredient followed by 7 empty blisters or by 7 dosage units that contain no active agent.

All publications cited in this specification, and the sequence listing, are incorporated herein by reference. While the invention has been described with reference to particular embodiments, it will be appreciated that modifications can be made without departing from the spirit of the invention. Such modifications are intended to fall within the scope of the appended claims.

1. A method of contraception that inhibits ovulation which comprises administering to a female of child bearing age over a period of 28 consecutive days:
(a) a first phase of from 21 to 27 daily dosage units of an active agent, each daily dosage unit containing an active agent consisting of a PR antagonist,
(b) a second phase of daily dosage units of 1 to 7 days of a pharmaceutically acceptable placebo,
the total of the daily dosage units being 28.

2. The method according to claim 1, wherein the PR antagonist is selected from the group consisting of mifepristone, onapristone, lilopristone, asoprisinil, CDB-2914, 5-(3,3-dimethyl-2-oxo-2,3-dihydro-1H-indol-5-yl)-1-methyl-1H-pyrrole-2-carbonitrile; 1-methyl-5-(2′-oxo-1′,2′-dihydrospiro[cyclobutane-1,3′-indol]-5′-yl)-1H-pyrrole-2-carbonitrile; 1-methyl-5-(2-oxo-2,3-dihydro-1H-indol-5-yl)-1H-pyrrole-2-carbonitrile; 5-(3-Ethyl-2-oxo-2,3-dihydro-1H-indol-5-yl)-1-methyl-1H-pyrrole-2-carbonitrile; 5-[(3R)-3-ethyl-2-oxo-2,3-dihydro-1H-indol-5-yl]-1-methyl-1H-pyrrole-2-carbonitrile; 5-[(3S)-3-ethyl-2-oxo-2,3-dihydro-1H-indol-5-yl]-1-methyl-1H-pyrrole-2-carbonitrile; 1-Methyl-5-(2′-oxo-1′,2′-dihydrospiro[cyclopropane-1,3′-indol]-5′-yl)-1H-pyrrole-2-carbonitrile; 5-[(3R)-3-ethyl-3-methyl-2-oxo-2,3-dihydro-1H-indol-5-yl]-1-methyl-1H-pyrrole-2-carbonitrile; 5-[(3S)-3-ethyl-3-methyl-2-oxo-2,3-dihydro-1H-indol-5-yl]-1-methyl-1H-pyrrole-2-carbonitrile; and 1-methyl-5-(1,3,3-trimethyl-2-oxo-2,3-dihydro-1H-indol-5-yl)-1H-pyrrole-2-carbonitrile, a compound of formula I: 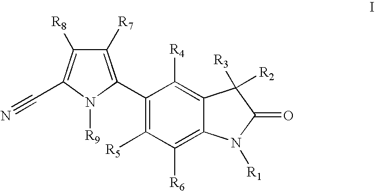
wherein:
R₁ is hydrogen, alkyl, substituted alkyl, cycloalkyl, C₃ to C₆ alkenyl, or C₃ to C₆ alkynyl;
R₂ and R₃ are each independently selected from the group consisting of hydrogen, alkyl, and substituted alkyl; or
R₂ and R₃ are taken together to form a ring and together contain —CH₂—(CH₂)ₙ—CH₂—; n is 0, 1, 2, or 3;
R₄ is hydrogen or halogen;
R₅ is hydrogen;
R₆ is hydrogen or halogen;
R₇ is hydrogen, alkyl, or halogen;
R₈ is hydrogen;
R₉ is hydrogen, alkyl, substituted alkyl, or COOR^{A}, where R^{A} is alkyl or substituted alkyl; and a compound of formula II: 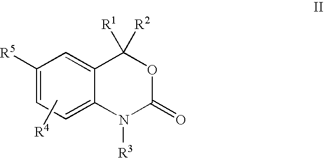
wherein:
R¹ and R² are independent substituents selected from the group consisting of H, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heterocyclic, substituted heterocyclic, COR^{A}, and NR^{B}COR^{A};
or R¹ and R² are fused to form:
a) a carbon-based 3 to 8 membered saturated spirocyclic ring;
b) a carbon-based 3 to 8 membered spirocyclic ring having in its backbone one or more carbon-carbon double bonds; or
c) a carbon-based 3 to 8 membered heterocyclic ring having in its backbone one to three heteroatoms selected from the group consisting of O, S and N;
the spirocyclic rings of a), b) and c) being optionally substituted by from 1 to 4 groups selected from the group consisting of fluorine, C₁ to C₆ alkyl, C₁ to C₆ alkoxy, C₁ to C₆ thioalkyl, CF₃, OH, CN, NH₂, NH(C₁ to C₆ alkyl), and N(C₁ to C₆ alkyl)₂;
R^{A} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;
R^{B} is H, C₁ to C₃ alkyl, or substituted C₁ to C₃ alkyl;
R³is H, OH, NH₂, C₁ to C₆ alkyl, substituted C₁ to C₆ alkyl, C₃ to C₆ alkenyl, substituted C₃ to C₆ alkenyl, alkynyl, substituted alkynyl, or COR^{C};
R^{C} is H, C₁ to C₄ alkyl, substituted C₁ to C₄ alkyl, aryl, substituted aryl, C₁ to C₄ alkoxy, substituted C₁ to C₄ alkoxy, C₁ to C₄ aminoalkyl, or substituted C₁ to C₄ aminoalkyl;
R⁴ is H, halogen, CN, NO₂, C₁ to C₆ alkyl, substituted C₁ to C₆ alkyl, alkynyl, substituted alkynyl, C₁ to C₆ alkoxy, substituted C₁ to C₆ alkoxy, amino, C₁ to C₆ aminoalkyl, or substituted C₁ to C₆ aminoalkyl;
R⁵is selected from the group consisting of (i) and (ii):
(i) a substituted benzene ring having the substituents X, Y and Z as shown below: 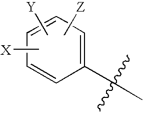
wherein:
X is selected from the group consisting of H, halogen, CN, C₁ to C₃ alkyl, substituted C₁ to C₃ alkyl, alkenyl, substituted alkenyl, alkynyl, substituted alkynyl, C₁ to C₃ alkoxy, substituted C₁ to C₃ alkoxy, C₁ to C₃ thioalkoxy, substituted C₁ to C₃ thioalkoxy, amino, C₁ to C₃ aminoalkyl, substituted C₁ to C₃ aminoalkyl, NO₂, C₁ to C₃ perfluoroalkyl, 5 or 6 membered heterocyclic ring containing in its backbone 1 to 3 heteroatoms selected from the group consisting of O, S, and N, COR^{D}, OCOR^{D}, and NR^{E}COR^{D};
R^{D} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;
R^{E} is H, C₁ to C₃ alkyl, or substituted C₁ to C₃ alkyl;
Y and Z are independent substituents selected from the group consisting of H, halogen, CN, NO₂, amino, aminoalkyl, C₁ to C₃ alkoxy, C₁ to C₄ alkyl, and C₁ to C₃ thioalkoxy;
wherein X, Y, and Z are not all H; and
(ii) a five or six membered ring having in its backbone 1, 2, or 3 heteroatoms selected from the group consisting of O, S, SO, SO₂ and NR⁶ and containing one or two independent substituents selected from the group consisting of H, halogen, CN, NO₂, amino, C₁ to C₄ alkyl, C₁ to C₃ alkoxy, C₁ to C₃ aminoalkyl, COR^{F}, and NR^{G}COR^{F};
R^{F} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;
R^{G} is H, C₁ to C₃ alkyl, or substituted C₁ to C₃ alkyl;
R⁶ is H, C₁ to C₃ alkyl, or C₁ to C₄ CO₂alkyl;
or pharmaceutically acceptable salt thereof.

3. A method of contraception that inhibits ovulation which comprises administering to a female of child bearing age over a period of 28 consecutive days:
(a) a first phase of from 21 to 27 daily dosage units of an active agent, each daily dosage unit containing an active agent consisting of a PR antagonist of the formula: 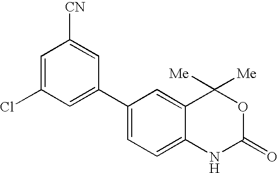
or a pharmaceutically acceptable salt thereof, and
(b) a second phase of daily dosage units of 1 to 7 days of a pharmaceutically acceptable placebo, the total of the daily dosage units being 28.

4. The method according to claim 3, which comprises:
(a) a first phase of 21 daily dosage units;
(b) a second phase of 7 daily dosage units of an orally and pharmaceutically acceptable placebo.

5. The method according to claim 3, which comprises:
(a) a first phase of 23 daily dosage units;
(b) a second phase of 5 daily dosage units of an orally and pharmaceutically acceptable placebo.

6. The method according to claim 3, which comprises:
(a) a first phase of 25 daily dosage units;
(b) a second phase of 3 daily dosage units of an orally and pharmaceutically acceptable placebo.

7. The method according to claim 3, which comprises:
(a) a first phase of 27 daily dosage units;
(b) a second phase of 1 daily dosage units of an orally and pharmaceutically acceptable placebo.

8. A method of contraception that inhibits ovulation which comprises administering to a female of child bearing age over a period of consecutive days:
(a) a first phase of from 21 to 27 daily dosage units of an active agent, each daily dosage unit containing an active agent consisting of a PR antagonist; and
(b) optionally a second phase of 1 to 7 days in which no effective amount of an active agent is administered, to total a period of consecutive days being 28 days.

9. The method according to claim 8, wherein the PR antagonist is selected from the group consisting of mifepristone, onapristone, lilopristone, asoprisinil, CDB-2914, 5-(3,3-dimethyl-2-oxo-2,3-dihydro-1H-indol-5-yl)-1-methyl-1H-pyrrole-2-carbonitrile; 1-methyl-5-(2′-oxo-1′,2′-dihydrospiro[cyclobutane-1,3′-indol]5′-yl)-1H-pyrrole-2-carbonitrile; 1-methyl-5-(2-oxo-2,3-dihydro-1H-indol-5-yl)-1H-pyrrole-2-carbonitrile; 5-(3-Ethyl-2-oxo-2,3-dihydro-1H-indol-5-yl)-1-methyl-1H-pyrrole-2-carbonitrile; 5-[(3R)-3-ethyl-2-oxo-2,3-dihydro-1H-indol-5-yl]-1-methyl-1H-pyrrole-2-carbonitrile; 5-[(3S)-3-ethyl-2-oxo-2,3-dihydro-1H-indol-5-yl]-1-methyl-1H-pyrrole-2-carbonitrile; 1-Methyl-5-(2′-oxo-1′,2′-dihydrospiro[cyclopropane-1,3′-indol]-5′-yl)-1H-pyrrole-2-carbonitrile; 5-[(3R)-3-ethyl-3-methyl-2-oxo-2,3-dihydro-1H-indol-5-yl]-1-methyl-1H-pyrrole-2-carbonitrile; 5-[(3S)-3-ethyl-3-methyl-2-oxo-2,3-dihydro-1H-indol-5-yl]-1-methyl-1H-pyrrole-2-carbonitrile; and 1-methyl-5-(1,3,3-trimethyl-2-oxo-2,3-dihydro-1H-indol-5-yl)-1H-pyrrole-2-carbonitrile, a compound of formula I: 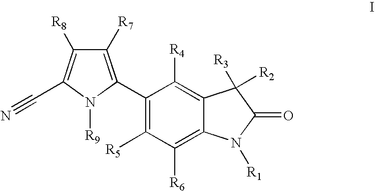
wherein:
R₁ is hydrogen, alkyl, substituted alkyl, cycloalkyl, C₃ to C₆ alkenyl, or C₃ to C₆ alkynyl;
R₂ and R₃ are each independently selected from the group consisting of hydrogen, alkyl, and substituted alkyl; or
R₂ and R₃ are taken together to form a ring and together contain —CH₂—(CH₂)ₙ—CH₂—; n is 0, 1, 2, or 3;
R₄ is hydrogen or halogen;
R₅ is hydrogen;
R₆ is hydrogen or halogen;
R₇ is hydrogen, alkyl, or halogen;
R₈ is hydrogen;
R₉ is hydrogen, alkyl, substituted alkyl, or COOR^{A}, where R^{A} is alkyl or substituted alkyl; and a compound of formula II: 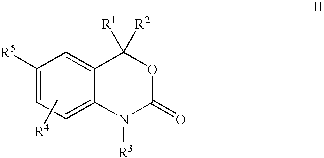
wherein:
R¹ and R² are independent substituents selected from the group consisting of H, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heterocyclic, substituted heterocyclic, COR^{A}, and NR^{B}COR^{A};
or R¹ and R² are fused to form:
a) a carbon-based 3 to 8 membered saturated spirocyclic ring;
b) a carbon-based 3 to 8 membered spirocyclic ring having in its backbone one or more carbon-carbon double bonds; or
c) a carbon-based 3 to 8 membered heterocyclic ring having in its backbone one to three heteroatoms selected from the group consisting of O, S and N;
the spirocyclic rings of a), b) and c) being optionally substituted by from 1 to 4 groups selected from the group consisting of fluorine, C₁ to C₆ alkyl, C₁ to C₆ alkoxy, C₁ to C₆ thioalkyl, CF₃, OH, CN, NH₂, NH(C₁ to C₆ alkyl), and N(C₁ to C₆ alkyl)₂;
R^{A} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;
R^{B} is H, C₁ to C₃ alkyl, or substituted C₁ to C₃ alkyl;
R³ is H, OH, NH₂, C₁ to C₆ alkyl, substituted C₁ to C₆ alkyl, C₃ to C₆ alkenyl, substituted C₃ to C₆ alkenyl, alkynyl, substituted alkynyl, or COR^{C};
R^{C} is H, C₁ to C₄ alkyl, substituted C₁ to C₄ alkyl, aryl, substituted aryl, C₁ to C₄ alkoxy, substituted C₁ to C₄ alkoxy, C₁ to C₄ aminoalkyl, or substituted C₁ to C₄ aminoalkyl;
R⁴ is H, halogen, CN, NO₂, C₁ to C₆ alkyl, substituted C₁ to C₆ alkyl, alkynyl, substituted alkynyl, C₁ to C₆ alkoxy, substituted C₁ to C₆ alkoxy, amino, C₁ to C₆ aminoalkyl, or substituted C₁ to C₆ aminoalkyl;
R⁵ is selected from the group consisting of (i) and (ii):
(i) a substituted benzene ring having the substituents X, Y and Z as shown below: 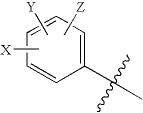
wherein:
X is selected from the group consisting of H, halogen, CN, C₁ to C₃ alkyl, substituted C₁ to C₃ alkyl, alkenyl, substituted alkenyl, alkynyl, substituted alkynyl, C₁ to C₃ alkoxy, substituted C₁ to C₃ alkoxy, C₁ to C₃ thioalkoxy, substituted C₁ to C₃ thioalkoxy, amino, C₁ to C₃ aminoalkyl, substituted C₁ to C₃ aminoalkyl, NO₂, C₁ to C₃ perfluoroalkyl, 5 or 6 membered heterocyclic ring containing in its backbone 1 to 3 heteroatoms selected from the group consisting of O, S, and N, COR^{D}, OCOR^{D}, and NR^{E}COR^{D};
R^{D} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;
R^{E} is H, C₁ to C₃ alkyl, or substituted C₁ to C₃ alkyl;
Y and Z are independent substituents selected from the group consisting of H, halogen, CN, NO₂, amino, aminoalkyl, C₁ to C₃ alkoxy, C₁ to C₄ alkyl, and C₁ to C₃ thioalkoxy;
wherein X, Y, and Z are not all H; and
(ii) a five or six membered ring having in its backbone 1, 2, or 3 heteroatoms selected from the group consisting of O, S, SO, SO₂ and NR⁶ and containing one or two independent substituents selected from the group consisting of H, halogen, CN, NO₂, amino, C₁ to C₄ alkyl, C₁ to C₃ alkoxy, C₁ to C₃ aminoalkyl, COR^{F}, and NR^{G}COR^{F};
R^{F} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;
R^{G} is H, C₁ to C₃ alkyl, or substituted C₁ to C₃ alkyl;
R⁶ is H, C₁ to C₃ alkyl, or C₁ to C₄ CO₂alkyl;
or pharmaceutically acceptable salt thereof.

10. A method of contraception that inhibits ovulation which comprises administering to a female of child bearing age over a period of consecutive days:
(a) a first phase of from 21 to 27 daily dosage units of an active agent, each daily dosage unit containing an active agent consisting of a PR antagonist having the formula: 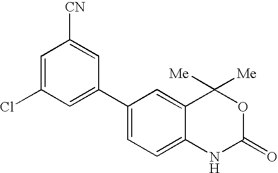
(b) optionally a second phase of 1 to 7 days in which no effective amount of an active agent is administered, to total period of consecutive days being 28 days.

11. A pharmaceutically useful kit adapted for daily oral administration, which comprises:
(a) 21 to 27 daily dosage units of an active agent, each daily dosage unit comprising an active agent containing an active agent consisting of a PR antagonist,
(b) 1 to 7 daily dosage units of a pharmaceutically acceptable placebo, wherein the total of the daily dosage units is 28; and
(c) one or more packages for said daily dosage units.

12. The pharmaceutically useful kit according to claim 11, wherein the PR antagonist is selected from the group consisting of mifepristone, onapristone, lilopristone, asoprisinil, CDB-2914, a compound of formula I: 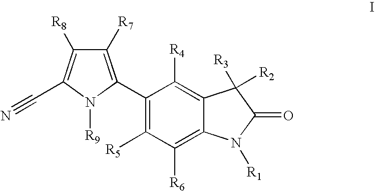
wherein:
R₁ is hydrogen, alkyl, substituted alkyl, cycloalkyl, C₃-C₆ alkenyl, or C₃-C₆ alkynyl;
R₂ and R₃ are each independently selected from the group consisting of hydrogen, alkyl, and substituted alkyl;
or R₂ and R₃ are taken together to form a ring —CH₂—(CH₂)ₙ—CH₂—;
n is 0, 1, or 2;
R₄ is hydrogen;
R₅ is hydrogen;
R₆ is hydrogen;
R₇ is hydrogen or alkyl;
R₈ is hydrogen;
R₉ is hydrogen, alkyl, substituted alkyl or COOR^{A};
R^{A} is alkyl or substituted alkyl; and a compound of formula II: 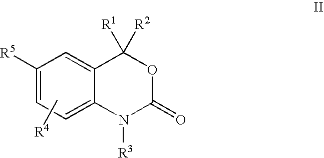
wherein:
R¹ and R² are independent substituents selected from the group consisting of H, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heterocyclic, substituted heterocyclic, COR^{A}, and NR^{B}COR^{A};
or R¹ and R² are fused to form:
a) a carbon-based 3 to 8 membered saturated spirocyclic ring;
b) a carbon-based 3 to 8 membered spirocyclic ring having in its backbone one or more carbon-carbon double bonds; or
c) a carbon-based 3 to 8 membered heterocyclic ring having in its backbone one to three heteroatoms selected from the group consisting of O, S and N;
the spirocyclic rings of a), b) and c) being optionally substituted by from 1 to 4 groups selected from the group consisting of fluorine, C₁ to C₆ alkyl, C₁ to C₆ alkoxy, C₁ to C₆ thioalkyl, CF₃, OH, CN, NH₂, NH(C₁ to C₆ alkyl), and N(C₁ to C₆ alkyl)₂;
R^{A} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;
R^{B} is H, C₁ to C₃ alkyl, or substituted C₁ to C₃ alkyl;
R³ is H, OH, NH₂, C₁ to C₆ alkyl, substituted C₁ to C₆ alkyl, C₃ to C₆ alkenyl, substituted C₃ to C₆ alkenyl, alkynyl, substituted alkynyl, or COR^{C};
R^{C} is H, C₁ to C₄ alkyl, substituted C₁ to C₄ alkyl, aryl, substituted aryl, C₁ to C₄ alkoxy, substituted C₁ to C₄ alkoxy, C₁ to C₄ aminoalkyl, or substituted C₁ to C₄ aminoalkyl;
R⁴ is H, halogen, CN, NO₂, C₁ to C₆ alkyl, substituted C₁ to C₆ alkyl, alkynyl, substituted alkynyl, C₁ to C₆ alkoxy, substituted C₁ to C₆ alkoxy, amino, C₁ to C₆ aminoalkyl, or substituted C₁ to C₆ aminoalkyl;
R⁵ is selected from the group consisting of (i) and (ii):
(i) a substituted benzene ring having the substituents X, Y and Z as shown below: 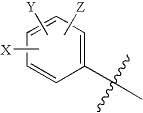
wherein:
X is selected from the group consisting of H, halogen, CN, C₁ to C₃ alkyl, substituted C₁ to C₃ alkyl, alkenyl, substituted alkenyl, alkynyl, substituted alkynyl, C₁ to C₃ alkoxy, substituted C₁ to C₃ alkoxy, C₁ to C₃ thioalkoxy, substituted C₁ to C₃ thioalkoxy, amino, C₁ to C₃ aminoalkyl, substituted C₁ to C₃ aminoalkyl, NO₂, C₁ to C₃ perfluoroalkyl, 5 or 6 membered heterocyclic ring containing in its backbone 1 to 3 heteroatoms selected from the group consisting of O, S, and N, COR^{D}, OCOR^{D}, and NR^{E}COR^{D};
R^{D} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;
R^{E} is H, C₁ to C₃ alkyl, or substituted C₁ to C₃ alkyl;
Y and Z are independent substituents selected from the group consisting of H, halogen, CN, NO₂, amino, aminoalkyl, C₁ to C₃ alkoxy, C₁ to C₄ alkyl, and C₁ to C₃ thioalkoxy;
wherein X, Y, and Z are not all H; and
(ii) a five or six membered ring having in its backbone 1, 2, or 3 heteroatoms selected from the group consisting of O, S, SO, SO₂ and NR⁶ and containing one or two independent substituents selected from the group consisting of H, halogen, CN, NO₂, amino, C₁ to C₄ alkyl, C₁ to C₃ alkoxy, C₁ to C₃ aminoalkyl, COR^{F}, and NR^{G}COR^{F};
R^{F} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;
R^{G} is H, C₁ to C₃ alkyl, or substituted C₁ to C₃ alkyl;
R⁶ is H, C₁ to C₃ alkyl, or C₁ to C₄ CO₂alkyl;
or pharmaceutically acceptable salt thereof.

13. The pharmaceutically useful kit adapted for daily oral administration according to claim 11, which comprises:
(a) 21 daily dosage units; and
(b) 7 daily dosage units of an orally and pharmaceutically acceptable placebo.

14. A pharmaceutically useful kit adapted for daily oral administration, which comprises:
(a) 21 to 27 daily dosage units of an active agent, each daily dosage unit comprising an active agent containing an active agent consisting of a PR antagonist having the formula: 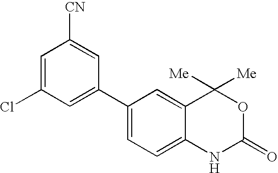
or a pharmaceutically acceptable salt thereof;
(b) 1 to 7 daily dosage units of a pharmaceutically acceptable placebo, wherein the total of the daily dosage units is 28; and
(c) one or more packages for said daily dosage units.

15. The pharmaceutically useful kit adapted for daily oral administration according to claim 14, which comprises:
(a) 21 daily dosage units; and
(b) 7 daily dosage units of an orally and pharmaceutically acceptable placebo.

16. The pharmaceutically useful kit adapted for daily oral administration according to claim 14, which comprises:
(a) 23 daily dosage units; and
(b) 5 daily dosage units of an orally and pharmaceutically acceptable placebo.

17. The pharmaceutically useful kit adapted for daily oral administration according to claim 14, which comprises:
(a) 25 daily dosage units; and
(b) 3 daily dosage units of an orally and pharmaceutically acceptable placebo.

18. The pharmaceutically useful kit adapted for daily oral administration according to claim 14, which comprises:
(a) 27 daily dosage units; and
(b) 1 daily dosage units of an orally and pharmaceutically acceptable placebo.

19. A pharmaceutically useful kit adapted for administration of a contraceptive regimen which comprises:
(a) 21 to 27 daily dosage units of an active agent adapted for delivery transdermal or mucosal delivery, said active agent consisting of a PR antagonist, and
(b) one or more packages for said daily dosage unit.

20. The pharmaceutically useful kit according to claim 19, wherein the PR antagonist is selected from the group consisting of mifepristone, onapristone, lilopristone, asoprisinil, CDB-2914, a compound of formula I: 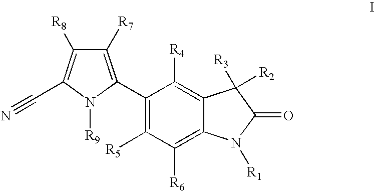
wherein:
R₁ is hydrogen, alkyl, substituted alkyl, cycloalkyl, C₃-C₆ alkenyl, or C₃-C₆ alkynyl;
R₂ and R₃ are each independently selected from the group consisting of hydrogen, alkyl, and substituted alkyl;
or R₂ and R₃ are taken together to form a ring —CH₂—(CH₂)ₙ—CH₂—;
n is 0, 1, or 2;
R₄ is hydrogen;
R₅ is hydrogen;
R₆ is hydrogen;
R₇ is hydrogen or alkyl;
R₈ is hydrogen;
R₉ is hydrogen, alkyl, substituted alkyl or COOR^{A};
R^{A} is alkyl or substituted alkyl; and a compound of formula II: 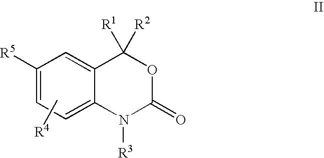
wherein:
R¹ and R² are independent substituents selected from the group consisting of H, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heterocyclic, substituted heterocyclic, COR^{A}, and NR^{B}COR^{A};
or R¹ and R² are fused to form:
a) a carbon-based 3 to 8 membered saturated spirocyclic ring;
b) a carbon-based 3 to 8 membered spirocyclic ring having in its backbone one or more carbon-carbon double bonds; or
c) a carbon-based 3 to 8 membered heterocyclic ring having in its backbone one to three heteroatoms selected from the group consisting of O, S and N;
the spirocyclic rings of a), b) and c) being optionally substituted by from 1 to 4 groups selected from the group consisting of fluorine, C₁ to C₆ alkyl, C₁ to C₆ alkoxy, C₁ to C₆ thioalkyl, CF₃, OH, CN, NH₂, NH(C₁ to C₆ alkyl), and N(C₁ to C₆ alkyl)₂;
R^{A} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;
R^{B} is H, C₁ to C₃ alkyl, or substituted C₁ to C₃ alkyl;
R³is H, OH, NH₂, C₁ to C₆ alkyl, substituted C₁ to C₆ alkyl, C₃ to C₆ alkenyl, substituted C₃ to C₆ alkenyl, alkynyl, substituted alkynyl, or COR^{C};
R^{C} is H, C₁ to C₄ alkyl, substituted C₁ to C₄ alkyl, aryl, substituted aryl, C₁ to C₄ alkoxy, substituted C₁ to C₄ alkoxy, C₁ to C₄ aminoalkyl, or substituted C₁ to C₄ aminoalkyl;
R⁴ is H, halogen, CN, NO₂, C₁ to C₆ alkyl, substituted C₁ to C₆ alkyl, alkynyl, substituted alkynyl, C₁ to C₆ alkoxy, substituted C₁ to C₆ alkoxy, amino, C₁ to C₆ aminoalkyl, or substituted C₁ to C₆ aminoalkyl;
R⁵ is selected from the group consisting of (i) and (ii):
(i) a substituted benzene ring having the substituents X, Y and Z as shown below: 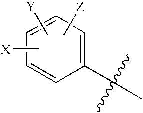
wherein:
X is selected from the group consisting of H, halogen, CN, C₁ to C₃ alkyl, substituted C₁ to C₃ alkyl, alkenyl, substituted alkenyl, alkynyl, substituted alkynyl, C₁ to C₃ alkoxy, substituted C₁ to C₃ alkoxy, C₁ to C₃ thioalkoxy, substituted C₁ to C₃ thioalkoxy, amino, C₁ to C₃ aminoalkyl, substituted C₁ to C₃ aminoalkyl, NO₂, C₁ to C₃ perfluoroalkyl, 5 or 6 membered heterocyclic ring containing in its backbone 1 to 3 heteroatoms selected from the group consisting of O, S, and N, COR^{D}, OCOR^{D}, and NR^{E}COR^{D};
R^{D} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;
R^{E} is H, C₁ to C₃ alkyl, or substituted C₁ to C₃ alkyl;
Y and Z are independent substituents selected from the group consisting of H, halogen, CN, NO₂, amino, aminoalkyl, C₁ to C₃ alkoxy, C₁ to C₄ alkyl, and C₁ to C₃ thioalkoxy;
wherein X, Y, and Z are not all H; and
(ii) a five or six membered ring having in its backbone 1, 2, or 3 heteroatoms selected from the group consisting of O, S, SO, SO₂ and NR⁶ and containing one or two independent substituents selected from the group consisting of H, halogen, CN, NO₂, amino, C₁ to C₄ alkyl, C₁ to C₃ alkoxy, C₁ to C₃ aminoalkyl, COR^{F}, and NR^{G}COR^{F};
R^{F} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;
R^{G} is H, C₁ to C₃ alkyl, or substituted C₁ to C₃ alkyl;
R⁶ is H, C₁ to C₃ alkyl, or C₁ to C₄ CO₂alkyl;
or pharmaceutically acceptable salt thereof.

21. A pharmaceutically useful kit adapted for administration of a contraceptive regimen which comprises:
(a) 1 to 27 daily dosage units of an active agent adapted for delivery transdermal or mucosal delivery, said active agent consisting of a PR antagonist having the formula: 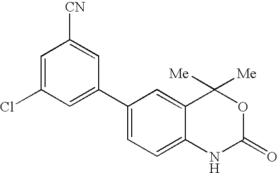
or a pharmaceutically acceptable salt thereof; and
(b) one or more packages for said daily dosage unit.
